(19) United States
(12) Patent Application Publication
(10) Pub. No.: US 2012/0141376 A1
(43) Pub. Date: Jun. 7, 2012

(54) PROSTATE SPECIFIC TRANSCRIPTS AND THE USE THEREOF FOR PROSTATE CANCER THERAPEUTICS AND DIAGNOSTICS

(76) Inventors: Richard Einstein, Gaithersburg, MD (US); Kevin Paul Kane, Alberta (CA); Matthew Paul Pando, Arlington, VA (US)

(21) Appl. No.: 12/309,478

(22) PCT Filed: Jun. 29, 2007
(86) PCT No.: PCT/EP2007/056584

§ 371 Date: Sep. 16, 2009

Related U.S. Application Data
(60) Provisional application No. 60/817,686, filed on Jul. 3, 2006.

(51) Int. Cl. A61K 51/08; C12Q 1/68; G01N 33/574; G01N 33/577; C07K 2/00; C07K 14/47; C07K 16/18; A61K 31/7088; A61K 38/02; A61P 35/00; A61K 38/43; A61K 38/18; A61K 51/04; A61K 39/00; A61P 37/04; A61K 39/395; C07H 21/04

(52) U.S. Cl. 424/1.69; 536/23.5; 536/24.33; 435/6.14; 435/6.11; 435/7.1; 435/7.92; 530/300; 530/350; 530/388.8; 530/391.3; 514/44 R; 514/19.5; 514/44 A; 424/94.3; 514/7.6; 424/1.73; 424/277.1; 424/155.1; 435/6.13

(57) ABSTRACT

Genes that are up regulated in human prostate tumor tissues and the corresponding proteins are identified. These genes and the corresponding antigens are suitable targets for the treatment, diagnosis or prophylaxis of prostate cancer.

### FIELD OF THE INVENTION

The present invention relates to the identification of DNA sequences that correspond to alternatively spliced events in genes expressed in prostate cancer cells. These genes or their corresponding proteins are to be targeted for the treatment, prevention and/or diagnosis of cancers wherein these genes are differentially regulated and/or spliced, particularly in prostate cancer.

### BACKGROUND OF THE INVENTION

Genetic detection of human disease states is a rapidly developing field (Taparowsky et al., 1982; Slamon et al., 1989; Sidransky et al., 1992; Miki et al., 1994; Dong et al., 1995; Morahan et al., 1996; Lifton, 1996; Barinaga, 1996). However, some problems exist with this approach. A number of known genetic lesions merely predispose an individual to the development of specific disease states. Individuals carrying the genetic lesion may not develop the disease state, while other individuals may develop the disease slate without possessing a particular genetic lesion. In human cancers, genetic defects may potentially occur in a large number of known tumor suppresser genes and proto-oncogenes.

Genetic detection of cancer has a long history. Some of the earliest genetic lesions shown to predispose to cancer were transforming point mutations in the ras oncogenes (Taparowsky et al., 1982). Transforming ras point mutations may be detected in the stool of individuals with benign and malignant colorectal tumors (Sidransky et al., 1992). However, only 50% of such tumors contained a ras mutation (Sidransky et al., 1992). Similar results have been obtained with amplification of HER-2/neu in breast and prostate cancer (Slamon et al., 1989), deletion and mutation of p53 in bladder cancer (Sidransky et al., 1991), deletion of DCC in colorectal cancer (Fearon et al., 1990) and mutation of BRCA1 in breast and prostate cancer (Miki et al., 1994).

None of these genetic lesions are capable of predicting a majority of individuals with cancer and most require direct sampling of a suspected tumor, and make screening difficult. Further, none of the markers described above are capable of distinguishing between metastatic and non-metastatic forms of cancer. In effective management of cancer patients, identification of those individuals whose tumors have already metastasized or are likely to metastasize is critical. Because metastatic cancer kills 560,000 people in the U.S. each year (ACS home page), identification of markers for metastatic prostate cancer would be an important advance.

A particular problem in cancer detection and diagnosis occurs with prostate cancer. Carcinoma of the prostate is the most frequently diagnosed cancer among men in the United States (Veltri et al., 1996). Prostate cancer was diagnosed in approximately 189,500 men in 1998 and about 40,000 men succumbed to the malignancy (Landis et al, 1998). Although relatively few prostate tumors progress to clinical significance during the lifetime of the patient, those which are progressive in nature are likely to have metastasized by the time of detection. Survival rates for individuals with metastatic prostate cancer are quite low. Between these extremes are patients with prostate tumors that will metastasize but have not yet done so, for whom surgical prostate removal is curative. Determination of which group a patient falls within is critical in determining optimal treatment and patient survival.

The FDA approval of the serum prostate specific antigen (PSA) test in 1984 changed the way that prostate disease was managed (Althoff et al., 1989; Cooner et al., 1990; Jacobson et al, 1995; Orozco et al., 1998). PSA is widely used as a serum biomarker to detect and monitor therapeutic response in prostate cancer patients (Badalament et al., 1996; O'Dowd et al., 1997). Several modifications in PSA assays (Partin and Oesterling, 1994; Babian et al., 1996; Zlotta et al, 1997) have resulted in earlier diagnoses and improved treatment.

Although PSA has been widely used as a clinical marker of prostate cancer since 1988 (Partin and Oesterling, 1994), screening programs utilizing PSA alone or in combination with digital rectal examination (DRE) have not been successful in improving the survival rate for men with prostate cancer (Partin and Oesterling, 1994). Although PSA is specific to prostate tissue, it is produced by normal and benign as well as malignant prostatic epithelium, resulting in a high false-positive rate for prostate cancer detection (Partin and Oesterling, 1994).

While an effective indicator of prostate cancer when serum levels are relatively high, PSA serum levels are more ambiguous indicators of prostate cancer when only modestly elevated, for example when levels are between 2-10 ng/ml. At these modest elevations, serum PSA may have originated from non-cancerous disease states such as BPH (benign prostatic hyperplasia), prostatitis or physical trauma (McCormack et al, 1995). Although application of the lower 2.0 ng/ml cancer detection cutoff concentration of serum PSA has increased the diagnosis of prostate cancer, especially in younger men with nonpalpable early stage tumors (Stage Tlc) (Soh et al., 1997; Carter and Coffey, 1997; Harris et al., 1997; Orozco et al., 1998), the specificity of the PSA assay for prostate cancer detection at low serum PSA levels remains a problem.

Several investigators have sought to improve upon the specificity of serologic detection of prostate cancer by examining a variety of other biomarkers besides serum PSA concentration (Ralph and Veltri, 1997). One of the most heavily investigated of these other biomarkers is the ratio of free versus total PSA (f/t PSA) in a patient's blood. Most PSA in serum is in a molecular form that is bound to other proteins such as alpha1-antichymotrypsin (ACT) or alpha2-macroglobulin (Christensson et al, 1993; Stenman et al., 1991; Lilja et al., 1991). Free PSA is not bound to other proteins. The ratio of free to total PSA (f/tPSA) is usually significantly higher in patients with BPH compared to those with organ confined prostate cancer (Marley et al., 1996; Oesterling et al., 1995; Pettersson et al., 1995). When an appropriate cutoff is determined for the f/tPSA assay, the f/tPSA assay can help distinguish patients with BPH from those with prostate cancer in cases in which serum PSA levels are only modestly elevated (Marley et al., 1996; Partin and Oesterling, 1996). Unfortunately, while f/tPSA may improve on the detection of prostate cancer, information in the f/tPSA ratio is insufficient to improve the sensitivity and specificity of serologic detection of prostate cancer to desirable levels.

Other markers that have been used for prostate cancer detection include prostatic acid phosphatase (PAP) and prostate secreted protein (PSP). PAP is secreted by prostate cells under hormonal control (Brawn et al., 1996). It has less specificity and sensitivity than does PSA. As a result, it is used much less now, although PAP may still have some applications for monitoring metastatic patients that have failed primary treatments. In general, PSP is a more sensitive biomarker than PAP, but is not as sensitive as PSA (Huang et al., 1993). Like PSA, PSP levels are frequently elevated in patients with BPH as well as those with prostate cancer.

Another serum marker associated with prostate disease is prostate specific membrane antigen (PSMA) (Horoszewicz et al., 1987; Carter and Coffey, 1996; Murphy et al., 1996). PSMA is a Type II cell membrane protein and has been identified as Folic Acid Hydrolase (FAH) (Carter and Coffey, 1996). Antibodies against PSMA react with both normal prostate tissue and prostate cancer tissue (Horoszewicz et al., 1987). Murphy et al. (1995) used ELISA to detect serum PSMA in advanced prostate cancer. As a serum test, PSMA levels are a relatively poor indicator of prostate cancer. However, PSMA may have utility in certain circumstances. PSMA is expressed in metastatic prostate tumor capillary beds (Silver et al., 1997) and is reported to be more abundant in the blood of metastatic cancer patients (Murphy et al., 1996). PSMA messenger RNA (mRNA) is down-regulated 8-10 fold in the LNCaP prostate cancer cell line after exposure to 5-alpha-dihydroxytestosterone (DHT) and is expressed at high levels in hormone deprived states (Israeli et al., 1994). Hormone modulation of PSMA expression may facilitate a clearer understanding of its regulation and significance in prostate cancer.

Two relatively new potential biomarkers for prostate cancer are human kallekrein 2 (HK2) (Piironen et al., 1996) and prostate specific transglutaminase (pTGase) (Dubbink et al., 1996). HK2 is a member of the kallekrein family that is secreted by the prostate gland (Piironen et al., 1996). Prostate specific transglutaminase is a calcium-dependent enzyme expressed in prostate cells that catalyzes post-translational cross-linking of proteins (Dubbink et al., 1996). In theory, serum concentrations of HK2 or pTGase may be of utility in prostate cancer detection or diagnosis, but the usefulness of these markers is still being evaluated.

Interleukin 8 (IL-8) has also been reported as a marker for prostate cancer. (Veltri et al., 1999). Serum IL-8 concentrations were reported to be correlated with increasing stage of prostate cancer and to be capable of differentiating BPH from malignant prostate tumors. (Id.) The wide-scale applicability of this marker for prostate cancer detection and diagnosis is still under investigation.

In addition to these protein markers for prostate cancer, several genetic changes have been reported to be associated with prostate cancer, including: allelic loss (Bova, et al., 1993; Macoska et al., 1994; Carter et al., 1990); DNA hypermethylation (Isaacs et al., 1994); point mutations or deletions of the retinoblastoma (Rb), p53 and KAI1 genes (Bookstein et al., 1990a; Bookstein et al., 1990b; Isaacs et al., 1991; Dong et al., 1995); and aneuploidy and aneusomy of chromosomes detected by fluorescence in situ hybridization (FISH) (Macoska et al., 1994; Visakorpi et al., 1994; Takahashi et al., 1994; Alcaraz et al., 1994). None of these have been reported to exhibit sufficient sensitivity and specificity to be useful as general screening tools for asymptomatic prostate cancer.

In current clinical practice, the scrum PSA assay and digital rectal exam (DRE) is used to indicate which patients should have a prostate biopsy (Lithrup et al., 1994; Orozco et al., 1998). Histological examination of the biopsied tissue is used to make the diagnosis of prostate cancer. Based upon the 189,500 cases of diagnosed prostate cancer in 1998 (Landis, 1998) and a known cancer detection rate of about 35% (Parker et al., 1996), it is estimated that in 1998 over one-half million prostate biopsies were performed in the United States (Orozco et al., 1998; Veltri et al., 1998). Clearly, there would be much benefit derived from a serological test that was sensitive enough to detect small and early stage prostate tumors that also had sufficient specificity to exclude a greater portion of patients with noncancerous or clinically insignificant conditions.

There remain deficiencies in the prior art with respect to the identification of the genes linked with the progression of prostate cancer and the development of diagnostic methods to monitor disease progression. Likewise, the identification of genes, which are differentially spliced and/or differentially expressed in prostate cancer, would be of considerable importance in the development of a rapid, inexpensive method to diagnose cancer. Although a few prostate specific genes have been cloned (PSA, PSMA, HK2, pTGase, etc.), these are typically not upregulated in prostate cancer. The identification of a novel, prostate specific transcript that is differentially expressed in prostate cancer, compared to non-malignant prostate tissue, would represent a major, unexpected advance for the diagnosis, prognosis and treatment of prostate cancer.

The CD8⁺ T lymphocyte subset can play an important role in immunosurveillance against tumorigenesis (Knutson et al., 2005). Peptides presented by major histocompatibility complex (MHC) class I molecules on diseased cells can be recognized by CD8⁺ T cells (Flutter et al., 2004). Activation of CD8⁺ T cells leading to their proliferation occurs via interaction with dendritic cells, specialized antigen presenting cells (APC), presenting peptide antigens. Following initial activation, CD8⁺ T cells are potent killer cells, capable of lysing cells expressing the activating peptide antigen they are specific for, when presented by self MHC molecules. The peptides presented by MHC class I molecules are derived from intracellular proteolytic processing events principally mediated by cytosolic multi-catalytic proteasome complexes (Guermonprez et al., 2005). Upon interaction of antigen specific T cell receptors with MHC/peptide antigen complexes, CD8⁺ cytolytic T lymphocytes (CTL) secrete the pore-forming protein perforin, and granzymes including granzyme B. These granule proteins coordinate proteolytic activation of the caspase cascade in target cells, leading to apoptotic death (Ashton-Richardt, 2005). There is great potential for the development of novel prostate cancer vaccines through the identification of prostate and prostate cancer specific transcripts and proteins, produced through alternative splicing.

Antigen presenting dendritic cell have been focused on for the development of cell-based tumor vaccine therapeutics. Dendritic cells are professional antigen presenting cells and are the most powerful activators of naïve T cells (Banchereau and Steinman, 1998; Fong and Engleman, 2000; Schuler et al., 2003). These cells can be mechanically and/or genetically manipulated with antigenic peptides, co-stimulatory molecules, and/or growth factors to create a population of cells specifically capable of activating CD8+ CTLs to specifically target tumor cells for destruction (Gabrilovich et al., 1996; Nair et al., 1997; Paglia et al., 1996; Zitvogel et al., 1996; Ashley et al., 1997; Mayordomo et al., 1996; Siders et al., 2003; Chen et al., 2003; Akiyama et al., 2000; Wan et al., 1997; Esslinger et al., 2002; Bonifaz et al., 2002). A variety of dendritic cell based cancer vaccines are being tested in clinical trials (Berzofsky et al., 2004). For example, 21 patients with recurrent or metastatic prostate cancer and elevated serum prostate acid phosphates (PAP) were treated with dendritic cells pulsed with rodent PAP. Ten of the treated patients developed T cell-proliferative responses to PAP (Fong et al., 2001). More recently, dentritic cell precursors have been exposed to PAP fusion proteins and then administered to patients to elicit a CTL response (Drugs R&D, 2006).

The use of therapeutic antibodies for treatment of cancers that target surface proteins is known. Examples thereof include RITUXAN® that targets CD20 on B cell lymphoma, Campath® that targets a surface antigen CD52 expressed by chronic lymphocytic leukemia, Herceptin® that targets erbB2 on breast and other cancers and Mybtara that targets CD33 surface antigen expressed on leukemia cells. However, to date, a monoclonal antibody for treatment of prostate cancer has not been approved for therapeutic use.

### SUMMARY OF THE INVENTION

The present invention relates to the identification of novel nucleic acid and amino acid sequences that are characteristic of prostate cancer cell or tissue, and which represent targets for therapy or diagnosis of such a condition in a subject.

The invention more specifically discloses 23 specific, isolated nucleic acid molecules that encode novel peptide sequences. These novel sequences were found to be differentially expressed between normal prostate and prostate cancer. These sequences and molecules represent targets and valuable information to develop methods and materials for the detection, diagnosis, and treatment of prostate cancer.

It is an object of the invention to provide methods and materials for treatment and diagnosis of prostate cancer.

It is a more specific object of the invention to identify novel exons (novel splice variants) that are expressed by prostate cancer tissue which are potential gene targets for treatment and diagnosis of prostate cancer.

It is a specific object of the invention to develop novel therapies for treatment of prostate cancer involving the administration or use of anti-sense oligonucleotides corresponding to novel gene targets that are specifically expressed by the prostate cancer.

It is another specific object of the invention to identify exons and the corresponding protein domain encoded by those exons specifically upregulated in prostate cancer cells.

It is another specific object of the invention to produce ligands that bind antigens encoded by the exons, expressed as a protein domain by certain prostate cancers, including, but not limited to, monoclonal antibodies.

It is another specific object of the invention to provide novel therapeutic regimens for the treatment of prostate cancer that involves the administration or use ofantigens expressed by certain prostate cancers, alone or in combination with adjuvants that elicit an antigen-specific cytotoxic T-cell lymphocyte response against cancer cells that express such antigen.

It is another object of the invention to provide novel therapeutic regimens for the treatment of prostate cancer that involves the administration or use of ligands, especially monoclonal antibodies that specifically bind novel antigens that are expressed by certain prostate cancers.

It is another object of this invention to provide pharmaceutical compositions comprising a ligand or antigen as defined above, in combination with a pharmaceutically acceptable carrier or excipient and/or an adjuvant.

It is another object of this invention to provide novel therapeutic regimens for the treatment of prostate cancer that involves the administration or use of cell based antitumor vaccination, especially antigen presenting dendritic cells directly loaded with peptides, by transduction with viral vectors expressing prostate tumor associated antigens, direct injection of prostate tumor-associated antigens, or naked DNA expression constructs.

It is another object of this invention to provide novel therapeutic regimens for the treatment of prostate cancer that involves the administration or use of cell based antitumor vaccination comprised of dendritic cells loaded with antigenic peptide as described above, which have been matured with bacterial LPS, TNFalpha, CD40 ligand, monocyte conditioning media, or cytokine cocktails.

It is another object of the invention to provide a novel method for diagnosis of prostate cancer by using ligands, e.g., monoclonal antibodies, which specifically bind to antigens that are specifically expressed by certain prostate cancers, in order to detect whether a subject has or is at increased risk of developing prostate cancer.

It is another object of the invention to provide a novel method of detecting persons having, or at increased risk of developing prostate cancer by use of labeled DNAs that hybridize to novel gene targets expressed by certain prostate cancers.

It is yet another object of the invention to provide diagnostic test kits for the detection of persons having or at increased risk of developing prostate cancer that comprise a ligand, e.g., monoclonal antibody that specifically binds to an antigen expressed by prostate cancer cells, and a detectable label, e.g. indicator enzymes, a radiolabels, fluorophores, or paramagnetic particles.

It is another object of the invention to provide diagnostic kits for detection of persons having or at risk of developing prostate cancer that comprise nucleic acid (e.g., DNA) primers or probes specific for novel gene targets specifically expressed by prostate cancer cells, and a detectable label, e.g. indicator enzymes, a radiolabels, fluorophores, or paramagnetic particles.

It is another object of this invention to provide methods for selecting, identifying, screening, characterizing or optimizing biologically active compounds, comprising a determination of whether a candidate compound binds, preferably selectively, an antigen or a polynucleotide as disclosed in the present application. Such compounds represent drug candidates or leads for treating cancer diseases, particularly prostate cancer.

It is another object of the invention to identify genes that are expressed in altered forms in prostate cancer cells. These forms represent splice variants of the gene, where the DATAS™ fragment either 1) indicates the splice event occurring within the gene, or 2) points to a gene that is actively spliced to produce different gene products. These different splice variants or isoforms can be targets for therapeutic intervention.

### DETAILED DESCRIPTION OF THE INVENTION

DATAS (Different Analysis of Transcripts with Alternative Splicing) analyzes structural differences between expressed genes and provides systematic access to alterations in RNA splicing (disclosed in U.S. Pat. No. 6,251,590, the disclosure of which is incorporated by reference in its entirety). Having access to these spliced sequences, which are critical for cellular homeostasis, represents a useful advance in functional genomics.

The DATAS Technology generates two libraries when comparing two samples, such as normal vs. tumor tissue. Each library specifically contains clones of sequences that are present and more highly expressed in one sample. For example, library A will contain sequences that are present in genes in the normal samples but absent in the tumor samples. These sequences are identified as being removed or spliced out from the genes in the tumor samples. In contrast, library B will contain sequences that are present only in the tumor samples and not present in the normal samples. These represent exons/introns that are alternatively spliced into genes expressed only in the tumor samples.

The present invention is based in part on the identification of exons that are isolated using DATAS and then determined to be differentially regulated or expressed in prostate tumor samples. Additionally, the invention provides exons that are prostate tumor specific or prostate specific that are not expressed on the cell surface, which may be useful in the preparation of prostate tumor vaccines, including prophylatic and therapeutic vaccines. Specifically, 23 expressed sequence tags were identified through DATAS and confirmed to be differentially expressed between normal prostate tissue and prostate tumor tissue. These DATAS fragments (DF) are small sections of genes that are selected for inclusion or exclusion in one sample but not the other. These small sections are part of the expressed gene transcript, and can consist of sequences derived from several different regions of the gene, including, but not limited to, portions of single exons, several exons, sequence from introns, and sequences from exons and introns. This alternative usage of exons in different biological samples produces different gene products from the same gene through a process well known in the art as alternative RNA splicing. In particular, 23 alternatively spliced isoforms have been identified from the DATAS fragment sequences, and produce alternate gene products that fit all the descriptions of targets and gene products below.

Alternatively spliced mRNA's produced from the same gene contain different ribonucleotide sequence, and therefore translate into proteins with different amino acid sequences. Nucleic acid sequences that are alternatively spliced into or out of the gene products can be inserted or deleted in frame or out of frame from the original gene sequence.

This leads to the translation of different proteins from each variant. Differences can include simple sequence deletions, or novel sequence information inserted into the gene product. Sequences inserted out of frame can lead to the production of an early stop codon and produce a truncated form of the protein. Alternatively, in-frame insertions of nucleic acid may cause an additional protein domain to be expressed from the mRNA. The end stage target is a novel protein containing either a novel epitope or function. Many variations of known genes have been identified and produce protein variants that can be agonistic or antagonistic with the original biological activity of the protein.

DATAS fragments thus identify genes and proteins which are subject to differential regulation and alternative splicing(s) in prostate cancer cells. DATAS fragments thus allow the definition of target molecules suitable for diagnosis or therapy of prostate cancers, which target molecules comprise all or a portion of genes or RNAs comprising the sequence of a DATAS fragment, or of genes or RNA from which the sequence of a DATAS fragment derives, as well as corresponding polypeptides or proteins, and variants thereof.

A particular object of this invention resides in isolated nucleic acid (e.g., DNA, RNA, etc) molecules (expressed by human prostate cancer cells) selected from the group consisting of:
(i) a nucleic acid comprising a sequence of any one of SEQ ID NOs: 1 to 23;
(ii) a variant of (i), wherein said variant has a nucleic acid sequence that is at least 70% identical to the sequence of (i) when aligned without allowing for gaps; and
(iii) a fragment of (i) or (ii) having a size of at least 20 nucleotides in length.

An other object of this invention resides in a polypeptide (antigen) (expressed by human prostate cancer cells), wherein said polypeptide is selected from the group consisting of:
(i) an antigen encoded by a nucleic acid sequence having at least 90% sequence identity in any one of SEQ ID NOs: 1 to 23;
(ii) an antigen derived from a protein comprising a sequence having at least 90% identity in any one of SEQ ID NOs: 1 to 23; and
(iii) an antigenic fragment of (i) or (ii).

A first type of target molecule is a target nucleic acid molecule comprising the sequence of a full gene or RNA molecule comprising the sequence of a DATAS fragment as disclosed in the present application. Indeed, since DATAS identifies genetic deregulations associated with prostate tumor, the whole gene or RNA sequence from which said DATAS fragment derives can be used as a target of therapeutic intervention or diagnosis.

Similarly, another type of target molecule is a target polypeptide molecule comprising the sequence of a full-length protein comprising the amino acid sequence encoded by a DATAS fragment as disclosed in the present application.

A further type of target molecule is a target nucleic acid molecule comprising a fragment of a gene or RNA as disclosed above. Indeed, since DATAS identifies genes and RNAs that are altered in prostate tumor cells, portions of such genes or RNAs, including portions that do not comprise the sequence of a DATAS fragment, can be used as a target for therapeutic intervention or diagnosis. Examples of such portions include: DATAS fragments, portions thereof, alternative exons or introns of said gene or RNA, exon-exon, exon-intron or intron-intron junction sequences generated by splicing(s) in said RNA, etc. Particular portions comprise a sequence encoding an extra-cellular domain of a polypeptide.

Similarly, another type of target molecule is a fragment of a protein comprising the amino acid sequence encoded by a DATAS fragment as disclosed in the present application. Such fragments may comprise or not the DATAS sequence, and may comprise newly generated amino acid sequence resulting, for instance, from a frame shift, a novel exon-exon or exon-intron junction, the creation of new stop codon, etc.

These target molecules (including genes, fragments, proteins and their variants) can serve as diagnostic agents and as targets for the development of therapeutics. For example, these therapeutics may modulate biological processes associated with prostate tumor viability. Agents may also be identified that are associated with the induction of apoptosis (cell death) in prostate tumor cells. Other agents can also be developed, such as monoclonal antibodies, which bind to the protein or its variant and alter the biological processes important for cell growth. Alternatively, antibodies can deliver a toxin which can inhibit cell growth and lead to cell death.

Specifically, the invention provides sequences that are expressed in a variant protein and are prostate tumor specific or prostate specific. These sequences are portions of genes identified to be in the plasma membrane of the cell through bioinformatic analysis, and the specific sequences of the invention are expressed on the extracellular region of the protein, so that the sequences may be useful in the preparation of prostate tumor vaccines, including prophylactic and therapeutic, cell and non-cell based vaccines.

Based thereon, it is anticipated that the disclosed genes that are associated with the differentially expressed sequences and the corresponding variant proteins should be suitable targets for prostate cancer therapy, prevention or diagnosis, e.g. for the development of antibodies, small molecular inhibitors, anti-sense therapeutics, and ribozymes. The potential therapies are described in greater detail below.

Such therapies will include the synthesis of oligonucleotides having sequences in the antisense orientation relative to the subject nucleic acids which appear to be up-regulated in prostate cancer. Suitable therapeutic antisense oligonucleotides will typically vary in length from two to several hundred nucleotides in length, more typically about 50-70 nucleotides in length or shorter. These antisense oligonucleotides may be administered as naked nucleic acids or in protected forms, e.g., encapsulated in liposomes. The use of liposomal or other protected forms may be advantageous as it may enhance in vivo stability and thus facilitate delivery to target sites, i.e., prostate tumor cells.

Also, the subject novel genes may be used to design novel ribozymes that target the cleavage of the corresponding mRNAs in prostate tumor cells. Similarly, these ribozymes may be administered in free (naked) form or by the use of delivery systems that enhance stability and/or targeting, e.g., liposomes.

Also, the present invention embraces the administration of use of nucleic acids that hybridize to the novel nucleic acid targets identified infra, attached to therapeutic effector moieties, e.g., radiolabels, (e.g., ⁹⁰Y, ¹³¹I) cytotoxins, cytotoxic enzymes, and the like in order to selectively target and kill cells that express these nucleic acids, i.e., prostate tumor cells.

Also, the present invention embraces the treatment and/or diagnosis of prostate cancer by targeting altered genes or the corresponding altered protein particularly splice variants that are expressed in altered form in prostate tumor cells. These methods will provide for the selective detection of cells and/or eradication of cells that express such altered forms thereby minimizing adverse effects to normal cells.

Still further, the present invention encompasses non-nucleic acid based therapies. For example, the invention encompasses the use of a DNA containing one of the novel cDNAs corresponding to novel antigen identified herein. It is anticipated that the antigens so encoded may be used as therapeutic or prophylactic anti-tumor vaccines. For example, a particular contemplated application of these antigens involves their administration with adjuvants that induce a cytotoxic T lymphocyte response.

Administration of the subject novel antigens in combination with an adjuvant may result in a humoral immune response against such antigens, thereby delaying or preventing the development of prostate cancer.

These embodiments of the invention will comprise administration of one or more of the subject novel prostate cancer antigens, ideally in combination with an adjuvant, e.g., PROVAX™, ISCOM'S®, DETOX®, SAF, Freund's adjuvant, Alum®, Saponin®, among others. This composition will be administered in an amount sufficient to be therapeutically or prophylactically effective, e.g. on the order of 50 to 20,000 mg/kg body weight, 100 to 5000 mg/kg body weight.

Additional embodiments of the invention will comprise the administration of antigen presenting dendritic cells loaded with novel antigenic peptides by direct mixing, transduction with viral vectors expressing prostate tumor associated antigens, direct injection of prostate tumor-associated antigens, or naked DNA expression constructs. These loaded dendritic cells may be matured with bacterial LPS, TNFalpha, CD40 ligand, monocyte conditioning media, or other cytokine cocktails prior to administration.

Yet another embodiment of the invention will comprise the preparation of monoclonal antibodies against the antigens encoded by the novel genes containing the nucleic acid sequences disclosed infra. Such monoclonal antibodies may be produced by conventional methods and include human monoclonal antibodies, humanized monoclonal antibodies, chimeric monoclonal antibodies, single chain antibodies, e.g., scFv's and antigen-binding antibody fragments such as Fab and Fab′ fragments. Methods for the preparation of monoclonal antibodies are known to those skilled in the art. In general, preparation of monoclonal antibodies will comprise immunization of an appropriate (non-homologous) host with the subject prostate cancer antigens, isolation of immune cells therefrom, use of such immune cells to isolate monoclonal antibodies and screening for monoclonal antibodies that specifically bind to either of such antigens. Antibody fragments may be prepared by known methods, e.g., enzymatic change of monoclonal antibodies.

These monoclonal antibodies and fragments will be useful for passive anti-tumor immunotherapy, or may be attached to therapeutic effector moieties, e.g., radiolabels, cytotoxins, therapeutic enzymes, agents that induce apoptosis, and the like in order to provide for targeted cytotoxicity, i.e., killing of human prostate tumor cells. Given the fact that the subject genes are apparently not significantly expressed by many normal tissues this should not result in significant adverse side effects (toxicity to non-target tissues).

In one embodiment, of the present invention such antibodies or fragments will be administered in labeled or unlabeled form, alone or in conjunction with other therapeutics, e.g., chemotherapeutics such as cisplatin, methotrexate, adriamycin, and the like suitable for prostate cancer therapy. The administered composition will also typically include a pharmaceutically acceptable carrier, and optionally adjuvants, stabilizers, etc., used in antibody compositions for therapeutic use.

Preferably, the subject monoclonal antibodies will bind the target antigens with high affinity, e.g., possess a binding affinity (Kd) on the order of 10⁻⁶ to 10⁻¹² M.

As noted, the present invention also embraces diagnostic applications that provide for detection of the expression of prostate specific splice variants disclosed herein. This will comprise detecting the expression of one or more of these genes at the RNA level and/or at the protein level.

For nucleic acids, expression of the subject genes will be detected by known nucleic acid detection methods, e.g., Northern blot hybridization, strand displacement amplification (SDA), catalytic hybridization amplification (CHA), and other known nucleic acid detection methods. Preferably, a cDNA library will be made from prostate cells obtained from a subject to be tested for prostate cancer by PCR using primers corresponding to the novel isoforms disclosed in this application.

The presence or absence of prostate cancer can be determined based on whether PCR products are obtained, and the level of expression. The levels of expression of such PCR product may be quantified in order to determine the prognosis of a particular prostate cancer patient (as the levels of expression of the PCR product often will increase or decrease significantly as the disease progresses.) This may provide a method for monitoring the status of a prostate cancer patient.

Alternatively, the status of a subject to be tested for prostate cancer may be evaluated by testing biological fluids, e.g., blood, urine, lymph, and the like with an antibody or antibodies or fragment that specifically binds to the novel prostate tumor antigens disclosed herein.

Methods for using antibodies to detect antigen expression are well known and include ELISA, competitive binding assays, and the like. In general, such assays use an antibody or antibody fragment that specifically binds the target antigen directly or indirectly bound to a label that provides for detection, e.g. indicator enzymes, a radiolabels, fiuorophores, or paramagnetic particles.

Patients which test positive for the enhanced presence of the antigen on prostate cells will be diagnosed as having or being at increased risk of developing prostate cancer. Additionally, the levels of antigen expression may be useful in determining patient status, i.e., how far disease has advanced (stage of prostate cancer).

As noted, the present invention provides novel splice variants that encode antigens that correlate to human prostate cancer. The present invention also embraces variants thereof. As used herein “variants” means sequences that are at least about 75% identical thereto, more preferably at least about 85% identical, and most preferably at least 90% identical and still more preferably at least about 95-99% identified when these DNA sequences are compared to a nucleic acid sequence encoding the subject DNAs or a fragment thereof having a size ofat least about 50 nucleotides. This includes allelic and splice variants of the subject genes. The present invention also encompasses nucleic acid sequences that hybridize to the subject splice variants under high, moderate or low stringency conditions e.g., as described infra.

Also, the present invention provides for primer pairs that result in the amplification of DNAs encoding the subject novel genes or a portion thereof in an mRNA library obtained from a desired cell source, typically human prostate cell or tissue sample. Typically, such primers will be on the order of 12 to 100 nucleotides in length, and will be constructed such that they provide for amplification of the entire or most of the target gene.

Also, the invention embraces the antigens encoded by the subject DNAs or fragments thereof that bind to or elicits antibodies specific to the full-length antigens. Typically, such fragments will be at least 10 amino acids in length, more typically at least 25 amino acids in length.

As noted, the subject DNA fragments are expressed in a majority of prostate tumor samples tested. The invention further contemplates the identification of other cancers that express such genes and the use thereof to detect and treat such cancers. For example, the subject DNA fragments or variants thereof may be expressed on other cancers, e.g., breast, ovary, pancreas, lung or prostate cancers. Essentially, the present invention embraces the detection of any cancer wherein the expression of the subject novel genes or variants thereof correlate to a cancer or an increased likelihood of cancer. To facilitate under-study of the invention, the following definitions are provided.

“Isolated tumor antigen or tumor protein” refers to any protein that is not in its normal cellular environment. This includes by way of example compositions comprising recombinant proteins encoded by the genes disclosed infra, pharmaceutical compositions comprising such purified proteins, diagnostic compositions comprising such purified proteins, and isolated protein compositions comprising such proteins. In preferred embodiments, an isolated prostate tumor protein according to the invention will comprise a substantially pure protein, in that it is substantially free of other proteins, preferably that is at least 90% pure, that comprises the amino acid sequence contained herein or natural homologues or mutants having essentially the same sequence. A naturally occurring mutant might be found, for instance, in tumor cells expressing a gene encoding a mutated protein according to the invention.

“Native tumor antigen or tumor protein” refers to a protein that is a non-human primate homologue of the protein having the amino acid sequence contained infra.

“Isolated prostate tumor gene or nucleic acid sequence” refers to a nucleic acid molecule that encodes a tumor antigen according to the invention which is not in its normal human cellular environment, e.g., is not comprised in the human or non-human primate chromosomal DNA. This includes by way of example vectors that comprise a gene according to the invention, a probe that comprises a gene according to the invention, and a nucleic acid sequence directly or indirectly attached to a detectable moiety, e.g. a fluorescent or radioactive label, or a DNA fusion that comprises a nucleic acid molecule encoding a gene according to the invention fused at its 5′ or 3′ end to a different DNA, e.g. a promoter or a DNA encoding a detectable marker or effector moiety. Also included are natural homologues or mutants having substantially the same sequence. Naturally occurring homologies that are degenerate would encode the same protein including nucleotide differences that do not change the corresponding amino acid sequence. Naturally occurring mutants might be found in tumor cells, wherein such nucleotide differences may result in a mutant tumor antigen. Naturally occurring homologues containing conservative substitutions are also encompassed.

“Variant of prostate tumor antigen or tumor protein” refers to a protein possessing an amino acid sequence that possess at least 90% sequence identity, more preferably at least 91% sequence identity, even more preferably at least 92% sequence identity, still more preferably at least 93% sequence identity, still more preferably at least 94% sequence identity, even more preferably at least 95% sequence identity, still more preferably at least 96% sequence identity, even more preferably at least 97% sequence identity, still more preferably at least 98% sequence identity, and most preferably at least 99% sequence identity, to the corresponding native tumor antigen wherein sequence identity is as defined infra. Preferably, this variant will possess at least one biological property in common with the native protein.

“Variant of prostate tumor gene or nucleic acid molecule or sequence” refers to a nucleic acid sequence that possesses at least 90% sequence identity, more preferably at least 91%, more preferably at least 92%, even more preferably at least 93%, still more preferably at least 94%, even more preferably at least 95%, still more preferably at least 96%, even more preferably at least 97%, even more preferably at least 98% sequence identity, and most preferably at least 99% sequence identity, to the corresponding native human nucleic acid sequence, wherein “sequence identity” is as defined infra.

“Fragment of prostate antigen encoding nucleic acid molecule or sequence” refers to a nucleic acid sequence corresponding to a portion of the native human gene wherein said portion is at least about 50 nucleotides in length, or 100, more preferably at least 150 nucleotides in length.

“Antigenic fragments of prostate tumor antigen” refer to polypeptides corresponding to a fragment of a prostate protein or a variant or homologue thereof that when used itself or attached to an immunogenic carrier elicits antibodies that specifically bind the protein. Typically such antigenic fragments will be at least 8-15 amino acids in length, and may be much longer.

Sequence identity or percent identity is intended to mean the percentage of the same residues shared between two sequences, referenced to human protein A or protein B or gene A or gene B, when the two sequences are aligned using the Clustal method [Higgins et al, Cabios 8:189-191 (1992)] of multiple sequence alignment in the Lasergene biocomputing software (DNASTAR, INC, Madison, Wis.), or alignment programs available from the Genetics Computer Group (GCG Wisconsin package, Accelrys, San Diego, Calif.). In this method, multiple alignments are carried out in a progressive manner, in which larger and larger alignment groups are assembled using similarity scores calculated from a series of pairwise alignments. Optimal sequence alignments are obtained by finding the maximum alignment score, which is the average of all scores between the separate residues in the alignment, determined from a residue weight table representing the probability of a given amino acid change occurring in two related proteins over a given evolutionary interval. Penalties for opening and lengthening gaps in the alignment contribute to the score. The default parameters used with this program are as follows: gap penalty for multiple alignment=10; gap length penalty for multiple alignment=10; k-tuple value in pairwise alignment=1; gap penalty in pairwise alignment=3; window value in pairwise alignment=5; diagonals saved in pairwise alignment=5. The residue weight table used for the alignment program is PAM25O [Dayhoff Et al., in Atlas of Protein Sequence and Structure, Dayhoff, Ed., NDRF, Washington, Vol. 5, suppl. 3, p. 345, (1978)].

Percent conservation is calculated from the above alignment by adding the percentage of identical residues to the percentage of positions at which the two residues represent a conservative substitution (defined as having a log odds value of greater than or equal to 0.3 in the PAM250 residue weight table). Conservation is referenced to human Gene A or gene B when determining percent conservation with non-human Gene A or gene B, e.g. mouse gene A or gene B, when determining percent conservation. Conservative amino acid changes satisfying this requirement include: R-K; E-D, Y-F, L-M; V-I, Q-H.

### Polypeptide Fragments

The invention provides polypeptide fragments of the disclosed proteins. Polypeptide fragments of the invention can comprise at least 8, more preferably at least 25, still more preferably at least 50 amino acid residues of the protein or an analogue thereof. More particularly such fragment will comprise at least 75, 100, 125, 150, 175, 200, 225, 250, 275 residues of the polypeptide encoded by the corresponding gene. Even more preferably, the protein fragment will comprise the majority of the native protein, e.g. about 100 contiguous residues of the native protein.

### Biologically Active Variants

The invention also encompasses mutants of the novel prostate proteins disclosed infra which comprise an amino acid sequence that is at least 80%, more preferably 90%, still more preferably 95-99% similar to the native protein.

Guidance in determining which amino acid residues can be substituted, inserted, or deleted without abolishing biological or immunological activity can be found using computer programs well known in the art, such as DNASTAR or software from the Genectics Computer Group (GCG). Preferably, amino acid changes in protein variants are conservative amino acid changes, i.e., substitutions of similarly charged or uncharged amino acids. A conservative amino acid change involves substitution of one of a family of amino acids which are related in their side chains. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cystine, serine, threonine, tyrosine) amino acids. Phenylalanine, tryptophan, and tyrosine are sometimes classified jointly as aromatic amino acids.

A subset of mutants, called muteins, is a group of polypeptides in which neutral amino acids, such as serines, are substituted for cysteine residues which do not participate in disulfide bonds. These mutants may be stable over a broader temperature range than native secreted proteins. See Mark et al., U.S. Pat. No. 4,959,314.

It is reasonable to expect that an isolated replacement of a leucine with an isoleucine or valine, an aspartate with a glutamate, a threonine with a serine, or a similar replacement of an amino acid with a structurally related amino acid will not have a major effect on the biological properties of the resulting secreted protein or polypeptide variant.

Protein variants include glycosylated forms, aggregative conjugates with other molecules, and covalent conjugates with unrelated chemical moieties. Also, protein variants also include allelic variants, species variants, and muteins. Truncations or deletions of regions which do not affect the differential expression of the gene are also variants. Covalent variants can be prepared by linking functionalities to groups which are found in the amino acid chain or at the N- or C-terminal residue, as is known in the art.

It will be recognized in the art that some amino acid sequence of the prostate proteins of the invention can be varied without significant effect on the structure or function of the protein. If such differences in sequence are contemplated, it should be remembered that there are critical areas on the protein which determine activity. In general, it is possible to replace residues that form the tertiary structure, provided that residues performing a similar function are used. In other instances, the type of residue may be completely unimportant if the alteration occurs at a non-critical region of the protein. The replacement of amino acids can also change the selectivity of binding to cell surface receptors. Ostade et al., Nature 361:266-268 (1993) describes certain mutations resulting in selective binding or TNF-alpha to only one of the two known types of TNF receptors. Thus, the polypeptides of the present invention may include one or more amino acid substitutions, deletions or additions, either from natural mutations or human manipulation.

The invention further includes variations of the prostate proteins disclosed infra which show comparable expression patterns or which include antigenic regions. Such mutants include deletions, insertions, inversions, repeats, and site substitutions. Guidance concerning which amino acid changes are likely to be phenotypically silent can be found in Bowie, J. U., et al., “Deciphering the Message in Protein Sequences: Tolerance to Amino Acid Substitutions,” Science 247:1306-1310 (1990).

Of particular interest are substitutions of charged amino acids with another charged amino acid and with neutral or negatively charged amino acids. The latter example results in proteins with reduced positive charge to improve the characteristics of the disclosed protein. The prevention of aggregation is highly desirable. Aggregation of proteins not only results in a loss of activity but can also be problematic when preparing pharmaceutical formulations, because they can be immunogenic. (Pinckard et al., Clin. Exp. Immunol. 2:331-340 (1967); Robbins et al., Diabetes 36:838-845 (1987); Cleland et al., Crit. Rev. Therapeutic Drug Carrier Systems 10:307-377 (1993)).

Amino acids in the polypeptides of the present invention that are essential for function can be identified by methods known in the art, such as site-directed mutagenesis or alanine-scanning mutagenesis (Cunningham and Wells, Science 244: 1081-1085 (1989)). The latter procedure introduces single alanine mutations at every residue in the molecule. The resulting mutant molecules are then tested for biological activity such as binding to a natural or synthetic binding partner. Sites that are critical for ligand-receptor binding can also be determined by structural analysis such as crystallization, nuclear magnetic resonance or photoaffinity labeling (Smith et al., J Mol. Biol. 224:899-904 (1992) and de Vos et al. Science 255: 306-312 (1992)).

As indicated, changes are preferably of a minor nature, such as conservative amino acid substitutions that do not significantly affect the folding or activity of the protein. Of course, the number of amino acid substitutions a skilled artisan would make depends on many factors, including those described above. Generally speaking, the number of substitutions for any given polypeptide will not be more than 50, 40, 30, 25, 20, 15, 10, 5 or 3.

### Fusion Proteins

Fusion proteins comprising proteins or polypeptide fragments of the subject prostate tumor antigen can also be constructed. Fusion proteins are useful for generating antibodies against amino acid sequences and for use in various assay systems. For example, fusion proteins can be used to identify proteins which interact with a protein of the invention or which interfere with its biological function. Physical methods, such as protein affinity chromatography, or library-based assays for protein-protein interactions, such as the yeast two-hybrid or phage display systems, can also be used for this purpose. Such methods are well known in the art and can also be used as drug screens. Fusion proteins comprising a signal sequence and/or a transmembrane domain of a protein according to the invention or a fragment thereof can be used to target other protein domains to cellular locations in which the domains are not normally found, such as bound to a cellular membrane or secreted extracellularly.

A fusion protein comprises two protein segments fused together by means of a peptide bond. As noted, these fragments may range in size from about 8 amino acids up to the full length of the protein.

The second protein segment can be a full-length protein or a polypeptide fragment. Proteins commonly used in fusion protein construction include β-galactosidase, β-glucuronidase, green fluorescent protein (GFP), autofluorescent proteins, including blue fluorescent protein (BFP), glutathione-S-transferase (GST), luciferase, horseradish peroxidase (HRP), and chloramphenicol acetyltransferase (CAT). Additionally, epitope tags can be used in fusion protein constructions, including histidine (His) tags, FLAG tags, influenza hemagglutinin (HA) tags, Myc tags, VSV-G tags, and thioredoxin (Trx) tags. Other fusion constructions can include maltose binding protein (MBP), S-tag, Lex a DNA binding domain (DBD) fusions, GAL4 DNA binding domain fusions, and herpes simplex virus (HSV) BP 16 protein fusions.

These fusions can be made, for example, by covalently linking two protein segments or by standard procedures in the art of molecular biology. Recombinant DNA methods can be used to prepare fusion proteins, for example, by making a DNA construct which comprises a coding sequence encoding a possible antigen according to the invention or a fragment thereof in proper reading frame with a nucleotide encoding the second protein segment and expressing the DNA construct in a host cell, as is known in the art. Many kits for constructing fusion proteins are available from companies that supply research labs with tools for experiments, including, for example, Promega Corporation (Madison, Wis.), Stratagene (La Jolla, Calif.), Clontech (Mountain View, Calif.), Santa Cruz Biotechnology (Santa Cruz, Calif.), MBL International Corporation (MIC; Watertown, Mass.), and Quantum Biotechnologies (Montreal, Canada; 1-888-DNA-KITS).

Proteins, fusion proteins, or polypeptides of the invention can be produced by recombinant DNA methods. For production of recombinant proteins, fusion proteins, or polypeptides, a sequence encoding the protein can be expressed in prokaryotic or eukaryotic host cells using expression systems known in the art. These expression systems include bacterial, yeast, insect, and mammalian cells.

The resulting expressed protein can then be purified from the culture medium or from extracts of the cultured cells using purification procedures known in the art. For example, for proteins fully secreted into the culture medium, cell-free medium can be diluted with sodium acetate and contacted with a cation exchange resin, followed by hydrophobic interaction chromatography. Using this method, the desired protein or polypeptide is typically greater than 95% pure. Further purification can be undertaken, using, for example, any of the techniques listed above.

It may be necessary to modify a protein produced in yeast or bacteria, for example by phosphorylation or glycosylation of the appropriate sites, in order to obtain a functional protein. Such covalent attachments can be made using known chemical or enzymatic methods.

A protein or polypeptide of the invention can also be expressed in cultured host cells in a form which will facilitate purification. For example, a protein or polypeptide can be expressed as a fusion protein comprising, for example, maltose binding protein, glutathione-S-transferase, or thioredoxin, and purified using a commercially available kit. Kits for expression and purification of such fusion proteins are available from companies such as New England BioLabs, Pharmacia, and Invitrogen. Proteins, fusion proteins, or polypeptides can also be tagged with an epitope, such as a “Flag” epitope (Kodak), and purified using an antibody which specifically binds to that epitope.

The coding sequence of the protein variants identified through the sequences disclosed herein can also be used to construct transgenic animals, such as mice, rats, guinea pigs, cows, goats, pigs, or sheep. Female transgenic animals can then produce proteins, polypeptides, or fusion proteins of the invention in their milk. Methods for constructing such animals are known and widely used in the art.

Alternatively, synthetic chemical methods, such as solid phase peptide synthesis, can be used to synthesize a secreted protein or polypeptide. General means for the production of peptides, analogs or derivatives are outlined in Chemistry and Biochemistry of Amino Acids, Peptides, and Proteins—A Survey of Recent Developments, B. Weinstein, ed. (1983). Substitution of D-amino acids for the normal L-stereoisomer can be carried out to increase the half-life of the molecule.

Typically, homologous polynucleotide sequences can be confirmed by hybridization under stringent conditions, as is known in the art. For example, using the following wash conditions: 2×SSC (0.3 M NaCl, 0.03 M sodium citrate, pH 7.0), 0.1% SDS, room temperature twice, 30 minutes each; then 2×SSC, 0.1% SDS, 50° C. once, 30 minutes; then 2×SSC, room temperature twice, 10 minutes each, homologous sequences can be identified which contain at most about 25-30% basepair mismatches. More preferably, homologous nucleic acid strands contain 15-25% basepair mismatches, even more preferably 5-15% basepair mismatches.

The invention also provides polynucleotide probes which can be used to detect complementary nucleotide sequences, for example, in hybridization protocols such as Northern or Southern blotting or in situ hybridizations. Polynucleotide probes of the invention comprise at least 12, 13, 14, 15, 16, 17, 18, 19, 20, 30, or 40 or more contiguous nucleotides of the nucleic acid sequences provided herein. Polynucleotide probes of the invention can comprise a detectable label, such as a radioisotopic, fluorescent, enzymatic, or chemiluminescent label.

Isolated genes corresponding to the cDNA sequences disclosed herein are also provided. Standard molecular biology methods can be used to isolate the corresponding genes using the cDNA sequences provided herein. These methods include preparation of probes or primers from the nucleotide sequence disclosed herein for use in identifying or amplifying the genes from mammalian, including human, genomic libraries or other sources of human genomic DNA.

Polynucleotide molecules of the invention can also be used as primers to obtain additional copies of the polynucleotides, using polynucleotide amplification methods. Polynucleotide molecules can be propagated in vectors and cell lines using techniques well known in the art. Polynucleotide molecules can be on linear or circular molecules. They can be on autonomously replicating molecules or on molecules without replication sequences. They can be regulated by their own or by other regulatory sequences, as is known in the art.

### Polynucleotide Constructs

Polynucleotide molecules comprising the coding sequences of the gene variants identified through the sequences disclosed herein can be used in a polynucleotide construct, such as a DNA or RNA construct. Polynucleotide molecules of the invention can be used, for example, in an expression construct to express all or a portion of a protein, variant, fusion protein, or single-chain antibody in a host cell. An expression construct comprises a promoter which is functional in a chosen host cell. The skilled artisan can readily select an appropriate promoter from the large number of cell type-specific promoters known and used in the art. The expression construct can also contain a transcription terminator which is functional in the host cell. The expression construct comprises a polynucleotide segment which encodes all or a portion of the desired protein. The polynucleotide segment is located downstream from the promoter. Transcription of the polynucleotide segment initiates at the promoter. The expression construct can be linear or circular and can contain sequences, if desired, for autonomous replication.

Also included are polynucleotide molecules comprising the promoter and UTR sequences of the subject novel genes, operably linked to the associated protein coding sequence and/or other sequences encoding a detectable or selectable marker. Such promoter and/or UTR-based constructs are useful for studying the transcriptional and translational regulation of protein expression, and for identifying activating and/or inhibitory regulatory proteins.

### Host Cells

An expression construct can be introduced into a host cell. The host cell comprising the expression construct can be any suitable prokaryotic or eukaryotic cell. Expression systems in bacteria include those described in Chang et al., Nature 275:615 (1978); Goeddel et al., Nature 281: 544 (1979); Goeddel et al., Nucleic Acids Res. 8:4057 (1980); EP 36,776; U.S. Pat. No. 4,551,433; deBoer et al., Proc. Natl. Acad. Sci. USA 80: 21-25 (1983); and Siebenlist et al., Cell 20: 269 (1980).

Expression systems in yeast include those described in Hinnnen et al., Proc. Natl. Acad. Sci. USA 75: 1929 (1978); Ito et al., J Bacterial 153: 163 (1983); Kurtz et al., Mol. Cell. Biol. 6: 142 (1986); Kunze et al., J Basic Microbiol. 25: 141 (1985); Gleeson et al., J. Gen. Microbial. 132: 3459 (1986), Roggenkamp et al., Mol. Gen. Genet. 202: 302 (1986)); Das et al., J. Bacteriol. 158: 1165 (1984); De Louvencourt et al., J Bacteriol. 154:737 (1983), Van den Berg et al., Bio/Technology 8: 135 (1990); Kunze et al., J Basic Microbiol. 25: 141 (1985); Cregg et al., Mol. Cell. Biol. 5: 3376 (1985); U.S. Pat. No. 4,837,148; U.S. Pat. No. 4,929,555; Beach and Nurse, Nature 300: 706 (1981); Davidow et al., Curr. Genet. 10: 380 (1985); Gaillardin et al., Curr. Genet. 10: 49 (1985); Ballance et al., Biochem. Biophys. Res. Commun. 112: 284-289 (1983); Tilburn et al., Gene 26: 205-22 (1983); Yelton et al., Proc. Natl. Acad, Sci. USA 81: 1470-1474 (1984); Kelly and Hynes, EMBO J. 4: 475479 (1985); EP 244,234; and WO 91/00357.

Expression of heterologous genes in insects can be accomplished as described in U.S. Pat. No. 4,745,051; Friesen et al. (1986) “The Regulation of Baculovirus Gene Expression” in: THE MOLECULAR BIOLOGY OF BACULOVIRUSES (W. Doerfler, ed.); EP 127,839; EP 155,476; Vlak et al., J. Gen. Virol. 69: 765-776 (1988); Miller et al., Ann. Rev. Microbial. 42: 177 (1988); Carbonell et al., Gene 73: 409 (1988); Maeda et al., Nature 315: 592-594 (1985); Lebacq-Verheyden et al., Mol. Cell. Biol. 8: 3129 (1988); Smith et al., Proc. Natl. Acad. Sci. USA 82: 8404 (1985); Miyajima et al., Gene 58: 273 (1987); and Martin et al., DNA 7:99 (1988). Numerous baculoviral strains and variants and corresponding permissive insect host cells from hosts are described in Luckow et al., Bio/Technology (1988) 6: 47-55, Miller et al., in GENETIC ENGINEERING (Setlow, J. K. et al. eds.), Vol. 8, pp. 277-279 (Plenum Publishing, 1986); and Maeda et al., Nature, 315: 592-594 (1985).

Mammalian expression can be accomplished as described in Dijkema et al., EMBO J. 4: 761 (1985); Gormanetal., Proc. Natl. Acad. Sci. USA 79: 6777 (1982b); Boshart et al., Cell 41: 521 (1985); and U.S. Pat. No. 4,399,216. Other features of mammalian expression can be facilitated as described in Ham and Wallace, Meth Enz. 58: 44 (1979);

Expression constructs can be introduced into host cells using any technique known in the art. These techniques include transferrin-polycation-mediated DNA transfer, transfection with naked or encapsulated nucleic acids, liposome-mediated cellular fusion, intracellular transportation of DNA-coated latex beads, protoplast fusion, viral infection, electroporation, “gene gun,” and calcium phosphate-mediated transfection.

The invention can also include hybrid and modified forms thereof including fusion proteins, fragments and hybrid and modified forms in which certain amino acids have been deleted or replaced, modifications such as where one or more amino acids have been changed to a modified amino acid or unusual amino acid.

Also included within the meaning of substantially homologous is any human or non-human primate protein which may be isolated by virtue of cross-reactivity with antibodies to proteins encoded by a gene described herein or whose encoding nucleotide sequences including genomic DNA, mRNA or cDNA may be isolated through hybridization with the complementary sequence of genomic or subgenomic nucleotide sequences or cDNA of a gene herein or fragments thereof. It will also be appreciated by one skilled in the art that degenerate DNA sequences can encode a tumor protein according to the invention and these are also intended to be included within the present invention as are allelic variants of the subject genes.

Preferred is a prostate protein according to the invention prepared by recombinant DNA technology. By “pure form” or “purified form” or “substantially purified form” it is meant that a protein composition is substantially free of other proteins which are not the desired protein.

The present invention also includes therapeutic or pharmaceutical compositions comprising a protein according to the invention in an effective amount for treating patients with disease, and a method comprising administering a therapeutically effective amount of the protein. These compositions and methods are useful for treating cancers associated with the subject proteins, e.g. prostate cancer. One skilled in the art can readily use a variety of assays known in the art to determine whether the protein would be useful in promoting survival or functioning in a particular cell type.

Anti-Prostate Antigen Antibodies

As noted, the invention includes the preparation and use of anti-prostate antigen antibodies and fragments for use as diagnostics and therapeutics. These antibodies may be polyclonal or monoclonal. Polyclonal antibodies can be prepared by immunizing rabbits or other animals by injecting antigen followed by subsequent boosts at appropriate intervals. The animals are bled and sera assayed against purified protein usually by ELISA or by bioassay based upon the ability to block the action of the corresponding gene. When using avian species, e.g., chicken, turkey and the like, the antibody can be isolated from the yolk of the egg. Monoclonal antibodies can be prepared after the method of Milstein and Kohler by fusing splenocytes from immunized mice with continuously replicating tumor cells such as myeloma or lymphoma cells. [Milstein and Kohler, Nature 256:495-497 (1975); Gulfre and Milstein, Methods in Enzymology: Immunochemical Techniques 73:1-46, Langone and Banatis eds., Academic Press, (1981) which are incorporated by reference]. The hybridoma cells so formed are then cloned by limiting dilution methods and supernates assayed for antibody production by ELISA, RIA or bioassay.

The unique ability of antibodies to recognize and specifically bind to target proteins provides an approach for treating an overexpression of the protein. Thus, another aspect of the present invention provides for a method for preventing or treating diseases involving overexpression of the protein by treatment of a patient with specific antibodies to the protein.

Specific antibodies, either polyclonal or monoclonal, to the protein can be produced by any suitable method known in the art as discussed above. For example, by recombinant methods, preferably in eukaryotic cells murine or human monoclonal antibodies can be produced by hybridoma technology or, alternatively, the protein, or an immunologically active fragment thereof, or an anti-idiotypic antibody, or fragment thereof can be administered to an animal to elicit the production of antibodies capable of recognizing and binding to the protein. Such antibodies can be from any class of antibodies including, but not limited to IgG, IgA, IgM, IgD, and IgE or in the case of avian species, IgY and from any subclass of antibodies.

The availability of isolated protein allows for the identification of small molecules and low molecular weight compounds that inhibit the binding of protein to binding partners, through routine application of high-throughput screening methods (HTS). HTS methods generally refer to technologies that permit the rapid assaying of lead compounds for therapeutic potential. HTS techniques employ robotic handling of test materials, detection of positive signals, and interpretation of data. Lead compounds may be identified via the incorporation of radioactivity or through optical assays that rely on absorbance, fluorescence or luminescence as read-outs. [Gonzalez, J. E. et al., Curr. Opin. Biotech. 9:624-63 1 (1998)].

Model systems are available that can be adapted for use in high throughput screening for compounds that inhibit the interaction of protein with its ligand, for example by competing with protein for ligand binding. Sarubbi et al., Anal. Biochem. 237:70-75 (1996) describe cell-free, non-isotopic assays for discovering molecules that compete with natural ligands for binding to the active site of IL-1 receptor. Martens, C. et al., Anal. Biochem. 273:20-31 (1999) describe a generic particle-based nonradioactive method in which a labeled ligand binds to its receptor immobilized on a particle; label on the particle decreases in the presence of a molecule that competes with the labeled ligand for receptor binding.

### Antibody Preparation

### (i) Starting Materials and Methods

Immunoglobulins (Ig) and certain variants thereof are known and many have been prepared in recombinant cell culture. For example, see U.S. Pat. No. 4,745,055; EP 256,654; EP 120,694; EP 125,023; EP 255,694; EP 266,663; WO 30 88/03559; Faulkner et al., Nature, 298: 286 (1982); Morrison, J. Immun., 123: 793 (1979); Koehler et al., Proc. Natl. Acad. Sci. USA, 77: 2197 (1980); Raso et al., Cancer Res., 41: 2073 (1981); Morrison et al., Ann. Rev. Immunol., 2: 239 (1984); Morrison, Science, 229: 1202 (1985); and Morrison et al., Proc. Natl. Acad. Sci. USA, 81: 6851 (1984). Reassorted immunoglobulin chains are also known. See, for example, U.S. Pat. No. 4,444,878; WO 88/03565; and EP 68,763 and references cited therein. The immunoglobulin moiety in the chimeras of the present invention may be obtained from IgG-1, IgG-2, IgG-3, or IgG-4 subtypes, IgA, IgE, IgD, or IgM, but preferably from IgG-1 or IgG-3.

### (ii) Polyclonal Antibodies

Polyclonal antibodies to the subject prostate antigens are generally raised in animals by multiple subcutaneous (sc) or intraperitoneal (ip) injections of the antigen and an adjuvant. It may be useful to conjugate the antigen or a fragment containing the target amino acid sequence to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde or succinic anhydride.

Animals are immunized against the polypeptide or fragment, immunogenic conjugates, or derivatives by combining about 1 mg or 1 μg of the peptide or conjugate (for rabbits or mice, respectively) with 3 volumes of Freund's complete adjuvant and injecting the solution intradermally at multiple sites. One month later the animals are boosted with 1/5 to 1/10 the original amount of peptide or conjugate in Freund's complete adjuvant by subcutaneous injection at multiple sites. Seven to 14 days later the animals are bled and the serum is assayed for antibody titer to the antigen or a fragment thereof. Animals are boosted until the titer plateaus. Preferably, the animal is boosted with the conjugate of the same polypeptide or fragment thereof, but conjugated to a different protein and/or through a different cross-linking reagent. Conjugates also can be made in recombinant cell culture as protein fusions. Also, aggregating agents such as alum are suitably used to enhance the immune response.

(iii) Monoclonal Antibodies

Monoclonal antibodies are obtained from a population of substantially homogeneous antibodies, i.e., the individual antibodies comprising the population are identical except for possible naturally occurring mutations that may be present in minor amounts. Thus, the modifier “monoclonal” indicates the character of the antibody as not being a mixture of discrete antibodies.

For example, monoclonal antibodies using for practicing this invention may be made using the hybridoma method first described by Kohler and Milstein, Nature, 256: 495 (1975), or may be made by recombinant DNA methods (Cabilly et al., supra).

In the hybridoma method, a mouse or other appropriate host animal, such as a hamster, is immunized as hereinabove described to elicit lymphocytes that produce or are capable of producing antibodies that will specifically bind to the antigen or fragment thereof used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 [Academic Press, 1986]).

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high-level production of antibody by the selected antibody-producing cells, and are sensitive to a medium such as HAT medium. Among these, preferred myeloma cell lines are murine myeloma lines, such as those derived from MOPC-21 and MPC-11 mouse tumors available from the Salk Institute Cell Distribution Center, San Diego, Calif. USA, and SP-2 cells available from the American Type Culture Collection, Rockville, Md. USA.

Culture medium in which hybridoma cells are growing is assayed for production of monoclonal antibodies directed against the prostate antigen. Preferably, the binding specificity of monoclonal antibodies produced by hybridoma cells is determined by immunoprecipitation or by an in vitro binding assay, such as radioimmunoassay (RIA) or enzyme-linked immunoabsorbent assay (ELISA).

The binding affinity of the monoclonal antibody can, for example, be determined by the Scatchard analysis of Munson and Pollard, Anal. Biochem., 107: 220 (1980).

After hybridoma cells are identified that produce antibodies of the desired specificity, affinity, and/or activity, the clones may be subcloned by limiting dilution procedures and grown by standard methods (Goding, supra). Suitable culture media for this purpose include, for example, D-MEM or RPMI-1640 medium. In addition, the hybridoma cells may be grown in vivo as ascites tumors in an animal.

The monoclonal antibodies secreted by the subdlones are suitably separated from the culture medium, ascites fluid, or serum by conventional immunoglobulin purification procedures such as, for example, protein A-Sepharose, hydroxyapatite chromatography, gel electrophoresis, dialysis, or affinity chromatography.

DNA encoding the monoclonal antibodies of the invention is readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA may be placed into expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. Review articles on recombinant expression in bacteria of DNA encoding the antibody include Skerra et al., Curr. Opinion in Immunol., 5: 256-262 (1993) and Pluckthun, Immunol. Revs., 130: 151-188 (1992).

The DNA also may be modified, for example, by substituting the coding sequence for human heavy- and light-chain constant domains in place of the homologous murine sequences (Morrison, et al., Proc. Natl. Acad. Sci. USA, 81: 6851 [1984]), or by covalently joining to the immunoglobulin coding sequence all or part of the coding sequence for a non-immunoglobulin polypeptide. In that manner, “chimeric” or “hybrid” antibodies are prepared that have the binding specificity of an anti-prostate antigen monoclonal antibody herein.

Typically such non-immunoglobulin polypeptides are substituted for the constant domains of an antibody of the invention, or they are substituted for the variable domains of one antigen-combining site of an antibody of the invention to create a chimeric bivalent antibody comprising one antigen-combining site having specificity for prostate antigen according to the invention and another antigen-combining site having specificity for a different antigen.

Chimeric or hybrid antibodies also may be prepared in vitro using known methods in synthetic protein chemistry, including those involving crosslinking agents. For example, immunotoxins may be constructed using a disulfide-exchange reaction or by forming a thioether bond. Examples of suitable reagents for this purpose include iminothiolate and methyl-4-mercaptobutyrimidate.

### (iv) Humanized Antibodies

Methods for humanizing non-human antibodies are well known in the art. Generally, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al., Nature 321, 522-525 [1986]; Riechmann et al., Nature 332, 323-327 [1988]; Verhoeyen et al., Science 239, 1534-1536 [1988]), by substituting rodent CDRs or CDR sequences for the corresponding sequences of a human antibody. Accordingly, such “humanized” antibodies are chimeric antibodies (Cabilly et al., supra), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence which is closest to that of the rodent is then accepted as the human framework (FR) for the humanized antibody (Sims et al., J. Immunol., 151: 2296 [1993]; Chothia and Lesk, J. Mol. Biol., 196: 901 [1987]). Another method uses a particular framework derived from the consensus sequence of all human antibodies of a particular subgroup of light or heavy chains. The same framework may be used for several different humanized antibodies (Carter et al., Proc. Natl. Acad. Sci. USA, 89: 4285 [1992]; Presta et al., J. Immunol., 151: 2623 [1993]).

It is further important that antibodies be humanized with retention of high affinity for the antigen and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the consensus and import sequences so that the desired antibody characteristic, such as increased affinity for the target antigen(s), is achieved. In general, the CDR residues are directly and most substantially involved in influencing antigen binding.

### (v) Human Antibodies

Human monoclonal antibodies can be made by the hybridoma method. Human myeloma and mouse-human heteromyeloma cell lines for the production of human monoclonal antibodies have been described, for example, by Kozbor, J. Immunol. 133, 3001 (1984); Brodeur, et al., Monoclonal Antibody Production Techniques and Applications, pp. 51-63 (Marcel Dekker, Inc., New York, 1987); and Boerner et al., J. Immunol., 147: 86-95 (1991).

It is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (J_{H}) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Natl. Acad. Sci. USA, 90: 2551 (1993); Jakobovits et al., Nature, 362: 255-258 (1993); Bruggermann et al., Year in Immuno., 7: 33 (1993).

Alternatively, the phage display technology (McCafferty et al., Nature, 348: 552-553 [1990]) can be used to produce human antibodies and antibody fragments in vitro, from immunoglobulin variable (V) domain gene repertoires from non-immunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B-cell. Phage display can be performed in a variety of formats; for their review see, e.g., Johnson and Chiswell, Curr. Op. Struct. Biol., 3: 564-571 (1993). Several sources of V-gene segments can be used for phage display. Clackson et al., Nature, 352: 624-628 (1991) isolated a diverse array of anti-oxazolone antibodies from a small random combinatorial library of V genes derived from the spleens of immunized mice. A repertoire of V genes from non-immunized human donors can be constructed and antibodies to a diverse array of antigens (including self-antigens) can be isolated essentially following the techniques described by Marks et al., J. Mol. Biol., 222: 581-597 (1991), or Griffith et al., EMBO J., 12: 725-734 (1993).

In a natural immune response, antibody genes accumulate mutations at a high rate (somatic hypermutation). Some of the changes introduced will confer higher affinity, and B cells displaying high-affinity surface immunoglobulin are preferentially replicated and differentiated during subsequent antigen challenge. This natural process can be mimicked by employing the technique known as “chain shuffling” (Marks et al., Bio/Technology, 10: 779-783 [1992]). In this method, the affinity of “primary” human antibodies obtained by phage display can be improved by sequentially replacing the heavy and light chain V region genes with repertoires of naturally occurring variants (repertoires) of V domain genes obtained from non-immunized donors. This technique allows the production of antibodies and antibody fragments with affinities in the nM range. A strategy for making very large phage antibody repertoires has been described by Waterhouse et al., Nucl. Acids Res., 21: 2265-2266 (1993).

Gene shuffling can also be used to derive human antibodies from rodent antibodies, where the human antibody has similar affinities and specificities to the starting rodent antibody. According to this method, which is also referred to as “epitope imprinting”, the heavy or light chain V domain gene of rodent antibodies obtained by phage display technique is replaced with a repertoire of human V domain genes, creating rodent-human chimeras. Selection on antigen results in isolation of human variable capable of restoring a functional antigen-binding site, i.e., the epitope governs (imprints) the choice of partner. When the process is repeated in order to replace the remaining rodent V domain, a human antibody is obtained (see PCT WO 93/06213, published Apr. 1, 1993). Unlike traditional humanization of rodent antibodies by CDR grafting, this technique provides completely human antibodies, which have no framework or CDR residues of rodent origin.

### (vi) Bispecific Antibodies

Bispecific antibodies are monoclonal, preferably human or humanized, antibodies that have binding specificities for at least two different antigens. In the present case, one of the binding specificities will be to a prostate antigen according to the invention. Methods for making bispecific antibodies are known in the art.

Traditionally, the recombinant production of bispecific antibodies is based on the co-expression of two immunoglobulin heavy chain-light chain pairs, where the two heavy chains have different specificities (Milstein and Cuello, Nature, 305: 537-539 [1983]). Because of the random assortment of immunoglobulin heavy and light chains, these hybridomas (quadromas) produce a potential mixture of 10 different antibody molecules, of which only one has the correct bispecific structure. The purification of the correct molecule, which is usually done by affinity chromatography steps, is rather cumbersome, and the product yields are low. Similar procedures are disclosed in WO 93/08829 published May 13, 1993, and in Traunecker et al., EMBO J., 10: 3655-3659 (1991).

According to a different and more preferred approach, antibody-variable domains with the desired binding specificities (antibody-antigencombining sites) are fused to immunoglobulin constant-domain sequences. The fusion preferably is with an immunoglobulin heavy-chain constant domain, comprising at least part of the hinge, CH2, and CH3 regions. It is preferred to have the first heavy-chain constant region (CH1), containing the site necessary for light-chain binding, present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the production of at least two polypeptide chains in equal ratios results in high yields or when the ratios are of no particular significance. In a preferred embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation.

For further details of generating bispecific antibodies, see, for example, Suresh et al., Methods in Enzymology, 121: 210 (1986).

(vii) Heteroconjuqate Antibodies

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (WO 91/00360; WO 92/00373; and EP 03089). Heteroconjugate antibodies may be made using any convenient cross-linking methods. Suitable cross-linking agents are well known in the art, and are disclosed in U.S. Pat. No. 4,676,980, along with a number of cross-linking techniques.

The polynucleotides and polypeptides of the present invention may be utilized in gene delivery vehicles. The gene delivery vehicle may be of viral or non-viral origin (see generally, Jolly, Cancer Gene Therapy 1:51-64 (1994); Kimura, Human Gene Therapy 5:845-852 (1994); Connelly, Human Gene Therapy 1:185-193 (1995); and Kaplitt, Nature Genetics 6:148-153 (1994)). Gene therapy vehicles for delivery of constructs including a coding sequence of a therapeutic according to the invention can be administered either locally or systemically. These constructs can utilize viral or non-viral vector approaches. Expression of such coding sequences can be induced using endogenous mammalian or heterologous promoters. Expression of the coding sequence can be either constitutive or regulated. Preferred vehicles for gene therapy include retroviral and adeno-viral vectors.

Representative examples of adenoviral vectors include those described by Berkner, Biotechniques 6:616-627 (Biotechniques); Rosenfeld et al., Science 252:431-434 (1991); WO 93/19191; Kolls et al., P.N.A.S. 215-219 (1994); Kass-Bisler et al., P.N.A.S. 90: 11498-11502 (1993); Guzman et al., Circulation 88: 2838-2848 (1993); Guzman et al., Cir. Res. 73: 1202-1207 (1993); Zabner et al., Cell 75: 207-216 (1993); Li et al., Hum. Gene Ther. 4: 403-409 (1993); Cailaud et al., Eur. J. Neurosci. 5: 1287-1291 (1993); Vincent et al., Nat. Genet. 5: 130-134 (1993); Jaffe et al., Nat. Genet. 1: 372-378 (1992); and Levrero et al., Gene 101: 195-202 (1992). Exemplary adenoviral gene therapy vectors employable in this invention also include those described in WO 94/12649, WO 93/03769; WO 93/19191; WO 94/28938; WO 95/11984 and WO 95/00655. Administration of DNA linked to kill adenovirus as described in Curiel, Hum. Gene Ther. 3: 147-154 (1992) may be employed.

Other gene delivery vehicles and methods may be employed; including polycationic condensed DNA linked or unlinked to kill adenovirus alone, for example Curiel, Hum. Gene Ther. 3: 147-154 (1992); ligand-linked DNA, for example see Wu, J. Biol. Chem. 264: 1.6985-16987 (1989); eukaryotic cell delivery vehicles cells, for example see U.S. Ser. No. 08/240,030, filed May 9, 1994, and U.S. Ser. No. 08/404,796; deposition of photopolymerized hydrogel materials; hand-held gene transfer particle gun, as described in U.S. Pat. No. 5,149,655; ionizing radiation as described in U.S. Pat. No. 5,206,152 and in WO 92/11033; nucleic charge neutralization or fusion with cell membranes. Additional approaches are described in Philip, Mol. Cell. Biol. 14:2411-2418 (1994), and in Woffendin, Proc. Natl. Acad. Sci. 91:1581-1585 (1994).

Naked DNA may also be employed. Exemplary naked DNA introduction methods are described in WO 90/11092 and U.S. Pat. No. 5,580,859. Uptake efficiency may be improved using biodegradable latex beads. DNA coated latex beads are efficiently transported into cells after endocytosis initiation by the beads. The method may be improved further by treatment of the beads to increase hydrophobicity and thereby facilitate disruption of the endosome and release of the DNA into the cytoplasm. Liposomes that can act as gene delivery vehicles are described in U.S. Pat. No. 5,422,120, PCT Patent Publication Nos. WO 95/13 796, WO 94/23697, and WO 91/14445, and EP No. 0 524 968.

Further non-viral delivery suitable for use includes mechanical delivery systems such as the approach described in Woffendin et al., Proc. Natl. Acad. Sci. USA 91(24): 11581-11585 (1994). Moreover, the coding sequence and the product of expression of such can be delivered through deposition of photopolymerized hydrogel materials. Other conventional methods for gene delivery that can be used for delivery of the coding sequence include, for example, use of hand-held gene transfer particle gun, as described in U.S. Pat. No. 5,149,655; use of ionizing radiation for activating transferred gene, as described in U.S. Pat. No. 5,206,152 and PCT Patent Publication No. WO 92/11033.

The subject antibodies or antibody fragments may be conjugated directly or indirectly to effective moieties, e.g., radionuclides, toxins, chemotherapeutic agents, prodrugs, cytoslatic agents, enzymes and the like. In a preferred embodiment the antibody or fragment will be attached to a therapeutic or diagnostic radiolabel directly or by use of a chelating agent. Examples of suitable radiolabels are well known and include ⁹⁰Y, ¹²⁵I, ¹³¹I, ¹¹¹I, ¹⁰⁵Rh, ¹⁵³Sm, ⁶⁷Cu, ⁶⁷Ga, ¹⁶⁶Ho, ¹⁷⁷Lu, ¹⁸⁶Re and ¹⁸⁸Re.

Examples of suitable drugs that may be coupled to antibodies include methotrexate, adriamycine and lymphokines such as interferons, interleukins and the like. Suitable toxins which may be coupled include ricin, cholera and diptheria toxin.

In a preferred embodiment, the subject antibodies will be attached to a therapeutic radiolabel and used for radioimmunotherapy.

Anti-Sense Oligonucleotides

In certain circumstances, it may be desirable to modulate or decrease the amount of the protein expressed by a prostate cell. Thus, in another aspect of the present invention, anti-sense oligonucleotides can be made and a method utilized for diminishing the level of expression a prostate antigen according to the invention by a cell comprising administering one or more anti-sense oligonucleotides. By anti-sense oligonucleotides reference is made to oligonucleotides that have a nucleotide sequence that interacts through base pairing with a specific complementary nucleic acid sequence involved in the expression of the target such that the expression of the gene is reduced. Preferably, the specific nucleic acid sequence involved in the expression of the gene is a genomic DNA molecule or mRNA molecule that encodes the gene. This genomic DNA molecule can comprise regulatory regions of the gene, or the coding sequence for the mature gene.

The term complementary to a nucleotide sequence in the context of antisense oligonucleotides and methods therefore means sufficiently complementary to such a sequence as to allow hybridization to that sequence in a cell, i.e., under physiological conditions. Antisense oligonucleotides preferably comprise a sequence containing from about 8 to about 100 nucleotides and more preferably the antisense oligonucleotides comprise from about 15 to about 30 nucleotides. Antisense oligonucleotides can also contain a variety of modifications that confer resistance to nucleolytic degradation such as, for example, modified internucleoside lineages [Uhlmann and Peyman, Chemical Reviews 90:543-548 (1990); Schneider and Banner, Tetrahedron Lett. 31:335, (1990) which are incorporated by reference], modified nucleic acid bases as disclosed in U.S. Pat. No. 5,958,773 and patents disclosed therein, and/or sugars and the like.

Any modifications or variations of the antisense molecule which are known in the art to be broadly applicable to antisense technology are included within the scope of the invention. Such modifications include preparation of phosphorus-containing linkages as disclosed in U.S. Pat. Nos. 5,536,821; 5,541,306; 5,550,111; 5,563,253; 5,571,799; 5,587,361, 5,625,050 and 5,958,773.

The antisense compounds of the invention can include modified bases. The antisense oligonucleotides of the invention can also be modified by chemically linking the oligonucleotide to one or more moieties or conjugates to enhance the activity, cellular distribution, or cellular uptake of the antisense oligonucleotide. Such moieties or conjugates include lipids such as cholesterol, cholic acid, thioether, aliphatic chains, phospholipids, polyamines, polyethylene glycol (PEG), palmityl moieties, and others as disclosed in, for example, U.S. Pat. Nos. 5,514,758, 5,565,552, 5,567,810, 5,574,142, 5,585,481, 5,587,371, 5,597,696 and 5,958,773.

Chimeric antisense oligonucleotides are also within the scope of the invention, and can be prepared from the present inventive oligonucleotides using the methods described in, for example, U.S. Pat. Nos. 5,013,830, 5,149,797, 5,403,711, 5,491,133, 5,565,350, 5,652,355, 5,700,922 and 5,958,773.

In the antisense art a certain degree of routine experimentation is required to select optimal antisense molecules for particular targets. To be effective, the antisense molecule preferably is targeted to an accessible, or exposed, portion of the target RNA molecule. Although in some cases information is available about the structure of target mRNA molecules, the current approach to inhibition using antisense is via experimentation. mRNA levels in the cell can be measured routinely in treated and control cells by reverse transcription of the mRNA and assaying the cDNA levels. The biological effect can be determined routinely by measuring cell growth or viability as is known in the art.

Measuring the specificity of antisense activity by assaying and analyzing cDNA levels is an art-recognized method of validating antisense results. It has been suggested that RNA from treated and control cells should be reverse-transcribed and the resulting cDNA populations analyzed. [Branch, A. D., T.I.B.S. 23:45-50 (1998)].

The therapeutic or pharmaceutical compositions of the present invention can be administered by any suitable route known in the art including for example intravenous, subcutaneous, intramuscular, transdermal, intrathecal or intracerebral. Administration can be either rapid as by injection or over a period of time as by slow infusion or administration of slow release formulation.

Additionally, the subject prostate tumor proteins can also be linked or conjugated with agents that provide desirable pharmaceutical or pharmacodynamic properties. For example, the protein can be coupled to any substance known in the art to promote penetration or transport across the blood-brain barrier such as an antibody to the transferrin receptor, and administered by intravenous injection (see, for example, Friden et al., Science 259:373-377 (1993) which is incorporated by reference). Furthermore, the subject protein A or protein B can be stably linked to a polymer such as polyethylene glycol to obtain desirable properties of solubility, stability, half-life and other pharmaceutically advantageous properties. [See, for example, Davis et al., Enzyme Eng. 4:169-73 (1978); Buruharn, Am. J. Hosp. Pharm. 51:210-218 (1994) which are incorporated by reference].

The compositions are usually employed in the form of pharmaceutical preparations. Such preparations are made in a manner well known in the pharmaceutical art. See, e.g. Remington Pharmaceutical Science, 18th Ed., Merck Publishing Co. Eastern PA, (1990). One preferred preparation utilizes a vehicle of physiological saline solution, but it is contemplated that other pharmaceutically acceptable carriers such as physiological concentrations of other non-toxic salts, five percent aqueous glucose solution, sterile water or the like may also be used. It may also be desirable that a suitable buffer be present in the composition. Such solutions can, if desired, be lyophilized and stored in a sterile ampoule ready for reconstitution by the addition of sterile water for ready injection. The primary solvent can be aqueous or alternatively non-aqueous. The subject prostate tumor antigens, fragments or variants thereof can also be incorporated into a solid or semi-solid biologically compatible matrix which can be implanted into tissues requiring treatment.

The carrier can also contain other pharmaceutically-acceptable excipients for modifying or maintaining the pH, osmolarity, viscosity, clarity, color, sterility, stability, rate of dissolution, or odor of the formulation. Similarly, the carrier may contain still other pharmaceutically-acceptable excipients for modifying or maintaining release or absorption or penetration across the blood-brain barrier. Such excipients are those substances usually and customarily employed to formulate dosages for parental administration in either unit dosage or multi-dose form or for direct infusion into the cerebrospinal fluid by continuous or periodic infusion.

Dose administration can be repeated depending upon the pharmacokinetic parameters of the dosage formulation and the route of administration used.

It is also contemplated that certain formulations containing the subject antibody or nucleic acid antagonists are to be administered orally. Such formulations are preferably encapsulated and formulated with suitable carriers in solid dosage forms. Some examples of suitable carriers, excipients, and diluents include lactose, dextrose, sucrose, sorbitol, mannitol, starches, gum acacia, calcium phosphate, alginates, calcium silicate, microcrystalline cellulose, polyvinylpyrrolidone, cellulose, gelatin, syrup, methyl cellulose, methyl- and propylhydroxybenzoates, talc, magnesium, stearate, water, mineral oil, and the like. The formulations can additionally include lubricating agents, wetting agents, emulsifying and suspending agents, preserving agents, sweetening agents or flavoring agents. The compositions may be formulated so as to provide rapid, sustained, or delayed release of the active ingredients after administration to the patient by employing procedures well known in the art. The formulations can also contain substances that diminish proteolytic degradation and promote absorption such as, for example, surface active agents.

The specific dose is calculated according to the approximate body weight or body surface area of the patient or the volume of body space to be occupied. The dose will also be calculated dependent upon the particular route of administration selected. Further refinement of the calculations necessary to determine the appropriate dosage for treatment is routinely made by those of ordinary skill in the art. Such calculations can be made without undue experimentation by one skilled in the art in light of the activity disclosed herein in assay preparations of target cells. Exact dosages are determined in conjunction with standard dose-response studies. It will be understood that the amount of the composition actually administered will be determined by a practitioner, in the light of the relevant circumstances including the condition or conditions to be treated, the choice of composition to be administered, the age, weight, and response of the individual patient, the severity of the patient's symptoms, and the chosen route of administration.

In one embodiment of this invention, the protein may be therapeutically administered by implanting into patients vectors or cells capable of producing a biologically-active form of the protein or a precursor of protein, i.e., a molecule that can be readily converted to a biological-active form of the protein by the body. In one approach, cells that secrete the protein may be encapsulated into semipermeable membranes for implantation into a patient. The cells can be cells that normally express the protein or a precursor thereof or the cells can be transformed to express the protein or a precursor thereof. It is preferred that the cell be of human origin and that the protein be a human protein when the patient is human. However, it is anticipated that non-human primate homologues of the protein discussed infra may also be effective.

Detection of Subject Prostate Proteins or Nucleic Acids

In a number of circumstances it would be desirable to determine the levels of protein or corresponding mRNA in a patient. Evidence disclosed infra suggests the subject prostate proteins may be expressed at different levels during some diseases, e.g., cancers, provides the basis for the conclusion that the presence of these proteins serves a normal physiological function related to cell growth and survival. Endogenously produced protein according to the invention may also play a role in certain disease conditions.

The term “detection” as used herein in the context of detecting the presence of protein in a patient is intended to include the determining of the amount of protein or the ability to express an amount of protein in a patient, the estimation of prognosis in terms of probable outcome of a disease and prospect for recovery, the monitoring of the protein levels over a period of time as a measure of status of the condition, and the monitoring of protein levels for determining a preferred therapeutic regimen for the patient, e.g. one with prostate cancer.

To detect the presence of a prostate protein according to the invention in a patient, a sample is obtained from the patient. The sample can be a tissue biopsy sample or a sample of blood, plasma, serum, CSF, urine or the like. It has been found that the subject proteins are expressed at high levels in some cancers. Samples for detecting protein can be taken from prostate tissues. When assessing peripheral levels of protein, it is preferred that the sample be a sample of blood, plasma or serum. When assessing the levels of protein in the central nervous system a preferred sample is a sample obtained from cerebrospinal fluid or neural tissue. The sample may be obtained by non-invasive methods, such as from tissue collection(s) or culture(s), or using directly available tissue material (urine, saliva, stools, hair, etc.).

In some instances, it is desirable to determine whether the gene is intact in the patient or in a tissue or cell line within the patient. By an intact gene, it is meant that there are no alterations in the gene such as point mutations, deletions, insertions, chromosomal breakage, chromosomal rearrangements and the like wherein such alteration might alter production of the corresponding protein or alter its biological activity, stability or the like to lead to disease processes, Thus, in one embodiment of the present invention a method is provided for detecting and characterizing any alterations in the gene. The method comprises providing an oligonucleotide that contains the gene, genomic DNA or a fragment thereof or a derivative thereof. By a derivative of an oligonucleotide, it is meant that the derived oligonucleotide is substantially the same as the sequence from which it is derived in that the derived sequence has sufficient sequence complementarily to the sequence from which it is derived to hybridize specifically to the gene. The derived nucleotide sequence is not necessarily physically derived from the nucleotide sequence, but may be generated in any manner including for example, chemical synthesis or DNA replication or reverse transcription or transcription.

Typically, patient genomic DNA is isolated from a cell sample from the patient and digested with one or more restriction endonucleases such as, for example, Taql and Alul.

Using the Southern blot protocol, which is well known in the art, this assay determines whether a patient or a particular tissue in a patient has an intact prostate gene according to the invention or a gene abnormality.

Hybridization to a gene would involve denaturing the chromosomal DNA to obtain a single-stranded DNA; contacting the single-stranded DNA with a gene probe associated with the gene sequence; and identifying the hybridized DNA-probe to detect chromosomal DNA containing at least a portion of a gene.

The term “probe” as used herein refers to a structure comprised of a polynucleotide that forms a hybrid structure with a target sequence, due to complementarily of probe sequence with a sequence in the target region. Oligomers suitable for use as probes may contain a minimum of about 8-12 contiguous nucleotides which are complementary to the targeted sequence and preferably a minimum of about 20.

A gene according to the present invention can be DNA or RNA oligonucleotides and can be made by any method known in the art such as, for example, excision, transcription or chemical synthesis. Probes may be labeled with any detectable label known in the art such as, for example, radioactive or fluorescent labels or enzymatic marker. Labeling of the probe can be accomplished by any method known in the art such as by PCR, random priming, end labeling, nick translation or the like. One skilled in the art will also recognize that other methods not employing a labeled probe can be used to determine the hybridization. Examples of methods that can be used for detecting hybridization include Southern blotting, fluorescence in situ hybridization, and single-strand conformation polymorphism with PCR amplification.

Hybridization is typically carried out at 25°-45° C., more preferably at 32°-40° C. and more preferably at 37°-38° C. The time required for hybridization is from about 0.25 to about 96 hours, more preferably from about one to about 72 hours, and most preferably from about 4 to about 24 hours.

Gene abnormalities can also be detected by using the PCR method and primers that flank or lie within the gene. The PCR method is well known in the art. Briefly, this method is performed using two oligonucleotide primers which are capable of hybridizing to the nucleic acid sequences flanking a target sequence that lies within a gene and amplifying the target sequence. The terms “oligonucleotide primer” as used herein refers to a short strand of DNA or RNA ranging in length from about 8 to about 30 bases. The upstream and downstream primers are typically from about 20 to about 30 base pairs in length and hybridize to the flanking regions for replication of the nucleotide sequence. The polymerization is catalyzed by a DNA-polymerase in the presence of deoxynucleotide triphosphates or nucleotide analogs to produce double-stranded DNA molecules. The double strands are then separated by any denaturing method including physical, chemical or enzymatic. Commonly, a method of physical denaturation is used involving heating the nucleic acid, typically to temperatures from about 80° C. to 105° C. for times ranging from about 1 to about 10 minutes. The process is repeated for the desired number of cycles.

The primers are selected to be substantially complementary to the strand of DNA being amplified. Therefore, the primers need not reflect the exact sequence of the template, but must be sufficiently complementary to selectively hybridize with the strand being amplified.

After PCR amplification, the DNA sequence comprising the gene or a fragment thereof is then directly sequenced and analyzed by comparison of the sequence with the sequences disclosed herein to identify alterations which might change activity or expression levels or the like.

In another embodiment, a method for detecting a tumor protein according to the invention is provided based upon an analysis of tissue expressing the gene. Certain tissues such as prostate tissues have been found to overexpress the subject gene. The method comprises hybridizing a polynucleotide to mRNA from a sample of tissue that normally expresses the gene. The sample is obtained from a patient suspected of having an abnormality in the gene.

To detect the presence of mRNA encoding the protein, a sample is obtained from a patient. The sample can be from blood or from a tissue biopsy sample. The sample may be treated to extract the nucleic acids contained therein. The resulting nucleic acid from the sample is subjected to gel electrophoresis or other size separation techniques.

The mRNA of the sample is contacted with a DNA sequence serving as a probe to form hybrid duplexes. The use of labeled probes as discussed above allows detection of the resulting duplex.

When using the cDNA encoding the protein or a derivative of the cDNA as a probe, high stringency conditions can be used in order to prevent false positives, that is the hybridization and apparent detection of the gene nucleotide sequence when in fact an intact and functioning gene is not present. When using sequences derived from the gene cDNA, less stringent conditions could be used, however, this would be a less preferred approach because of the likelihood of false positives. The stringency of hybridization is determined by a number of factors during hybridization and during the washing procedure, including temperature, ionic strength, length of time and concentration of formamide. These factors are outlined in, for example, Sambrook et al. [Sambrook et al. (1989), supra].

In order to increase the sensitivity of the detection in a sample of mRNA encoding the protein A or protein B, the technique of reverse transcription/polymerization chain reaction (RT/PCR) can be used to amplify cDNA transcribed from mRNA encoding the prostate tumor antigen. The method of RT/PCR is well known in the art, and can be performed as follows. Total cellular RNA is isolated by, for example, the standard guanidium isothiocyanate method and the total RNA is reverse transcribed. The reverse transcription method involves synthesis of DNA on a template of RNA using a reverse transcriptase enzyme and a 3′ end primer. Typically, the primer contains an oligo(dT) sequence. The cDNA thus produced is then amplified using the PCR method and gene A or gene B specific primers. [Belyaysky et al., Nucl. Acid Res. 17:2919-2932 (1989); Krug and Berger, Methods in Enzymology, 152:316-325, Academic Press, NY (1987) which are incorporated by reference].

The polymerase chain reaction method is performed as described above using two oligonucleotide primers that are substantially complementary to the two flanking regions of the DNA segment to be amplified. Following amplification, the PCR product is then electrophoresed and detected by ethidium bromide staining or by phosphoimaging.

The present invention further provides for methods to detect the presence of the protein in a sample obtained from a patient. Any method known in the art for detecting proteins can be used. Such methods include, but are not limited to immunodiffusion, immunoelectrophoresis, immunochemical methods, binder-ligand assays, immunohistochemical techniques, agglutination and complement assays. [Basic and Clinical Immunology, 217-262, Sites and Terr, eds., Appleton & Lange, Norwalk, Conn., (1991), which is incorporated by reference]. Preferred are binder-ligand immunoassay methods including reacting antibodies with an epitope or epitopes of the prostate tumor antigen protein and competitively displacing a labeled prostate antigen according to the invention or derivative thereof.

As used herein, a derivative of the subject prostate tumor antigen is intended to include a polypeptide in which certain amino acids have been deleted or replaced or changed to modified or unusual amino acids wherein the derivative is biologically equivalent to gene and wherein the polypeptide derivative cross-reacts with antibodies raised against the protein. By cross-reaction it is meant that an antibody reacts with an antigen other than the one that induced its formation.

Numerous competitive and non-competitive protein binding immunoassays are well known in the art. Antibodies employed in such assays may be unlabeled, for example as used in agglutination tests, or labeled for use in a wide variety of assay methods. Labels that can be used include radionuclides, enzymes, fluorescers, chemiluminescers, enzyme substrates or co-factors, enzyme inhibitors, particles, dyes and the like for use in radioimmunoassay (RIA), enzyme immunoassays, e.g., enzyme-linked immunosorbent assay (ELISA), fluorescent immunoassays and the like.

A further aspect of this invention relates to a method for selecting, identifying, screening, characterizing or optimizing biologically active compounds, comprising a determination of whether a candidate compound binds, preferably selectively, a target molecule as disclosed above. Such target molecules include nucleic acid sequences, polypeptides and fragments thereof, typically prostate-specific antigens, even more preferably extracellular portions thereof. Binding may be assessed in vitro or in vivo, typically in vitro, in cell based or cell free systems. Typically, the target molecule is contacted with the candidate compound in any appropriate device, and the formation of a complex is determined. The target molecule and/or the candidate compound may be immobilized on a support. The compounds identified or selected represent drug candidates or leads for treating cancer diseases, particularly prostate cancer.

While the invention has been described supra, including preferred embodiments, the following examples are provided to further illustrate the invention.

### Example

### Tissue Sources

Appropriate patient samples were procured for evaluation of research protocol. Samples were provided with relevant clinical parameters, and patient consent. Histological assessment was performed on all samples and diagnosis by pathology confirmed the presence and/or absence of malignancy within each sample. Clinical data generally included patent history, physiopathology, and parameters relating to prostate cancer physiology. Ten normal and ten malignant samples were procured along with available clinical information. In addition, ten samples from organs other than normal prostate and prostate cancer were procured to determine the tissue specific expression profile of epitopes. RNA derived from normal tissue samples was obtained from known commercial sources.

Additional patient samples were procured for the cytotoxic T Lymphocyte (CTL) assay. Peripheral blood monocytes (PBMC) were isolated from blood drawn from patients with relevant clinical parameters, and with patient consent. Clinical data generally included patent history, physiopathology, and parameters relating to prostate cancer physiology. PBMCs were collected from 6 prostate cancer patients that met the study requirements. Duplicate samples were obtained from a single patient.

### Generation of the DATAS Library;

Samples were pooled based on their pathological diagnosis (normal vs. tumor). Samples were pooled based on equivalent amounts of total RNA to produce total pooled RNA samples of 100 ug. DATAS libraries were constructed as previously disclosed in U.S. Pat. No. 6,251,590, the disclosure of which is incorporated by reference in its entirety. Briefly, total RNA was isolated from the normal and tumor pooled samples and mRNA was subsequently purified from the total RNA for each pooled sample. Synthesis of cDNA was performed using a biotinylated oligo (dT) primer. The biotinylated cDNA was hybridized with the mRNA of the opposite sample to form heteroduplexes between the cDNA and the mRNA. For example, the biotinylated cDNA of the pooled normal prostate sample was hybridized with prostate tumor mRNA. Similarly, prostate tumor biotinylated cDNA was hybridized with prostate normal RNA to generate the second DATAS library. Streptavidin coated beads were used to purify the complexes by binding the biotin present on the cDNA. The heteroduplexes were digested with RNAse H to degrade the RNA that was complementary to the cDNA. All mRNA sequences that were different from the cDNA remained intact. These single stranded RNA fragments or “loops” were subsequently amplified with degenerate primers and cloned into either pGEM-Tor pCR II TOPO vector (Company source) to produce the DATAS library.

### Clone Sequencing and Bioinformatics Analysis;

The DATAS library was used to transform E. coli so that individual clones could be isolated using standard molecular biology techniques. From these libraries, 10,665 individual clones were isolated and sequenced using an automated Applied Biosystems 3100 sequencer. The nucleotide sequences that were obtained were submitted to the bioinformatics pipeline tor analysis. As the DATAS library is prepared with PCR amplified DNA, many copies of the same sequence are present in the clones isolated from the libraries. Therefore it is important to reduce the redundancy of the clones to identify the number of unique, nonrepeating sequences that are isolated. From this large set of DATAS fragments, 1699 unique, nonredundant sequences were identified and each DATAS fragment was annotated with a candidate gene. The annotation was performed by aligning the DATAS fragment to the human genome sequence by two methods; 1) a publicly available alignment and genome viewer tool, Blat (Kent et al., 2002); and 2) a commercially available genomic alignment andviewer tool, Prophecy (Doubletwist). Each DATAS fragment sequence was annotated with a corresponding gene that overlapped the genomic sequence containing the DATAS fragment. Genes were annotated with either the RefSeq accession number, or a hypothetical gene prediction from different algorithms, for example, Genscan, Twinscan, or Fgenesh++. Identified genes were either matched to the sequence of the DATAS fragment (in case of exon to fragment match), or overlapped with the DATAS fragment (in case of intron to fragment match), and the full length sequence of the gene was identified. These sequences were further analyzed to detect all potential membrane spanning proteins. Membrane proteins were predicted through the use of different algorithms publicly available. For example, TMHMM (CBS) was used to identify membrane-spanning domains present within the amino acid sequence of the candidate gene. DATAS fragments were located within the sequence in an attempt to determine whether the spliced event affected intracellular or extraceullar domains. Genes associated with the sequence were ranked in order to maximize the identification of successful therapeutic targets.

### Monoclonal Antibody Target Selection:

The highest priority genes for antibody therapeutics development had characteristics where the gene was a known membrane protein, the function of the gene was known, and the DATAS fragment mapped to an intron on the extracellular domain of the protein, indicating that the DATAS fragment would be presented outside the cell, and available for therapeutic intervention by monoclonal antibodies.

Based on the bioinformatic analysis, clones relevant to monoclonal antibody therapeutics development were prioritized.

### CTL Epitope Identification:

The highest priority DATAS fragments for inclusion in the CTL screening assay were those that contained novel sequence information, such as those containing novel exons or exon extensions. Preferred DATAS fragment clones are linked to cancer initiation or progression and encode a cytoplasmic protein.

The 192 selected DATAS fragments were PCR amplified out of the library vector and directionally cloned into a eukaryotic expression vector down stream from staggered start site initiation codons. This configuration allows the DATAS fragment to be expressed in all the open reading frames, which is required because the correct reading frame is not always readily apparent.

Plasmid preparations were performed and the expression vectors containing the selcted DATAS fragment clones were re-arrayed into 96 well plates, which were subsequently used to spot glass slides for use in the CTL assay previously disclosed in International Patent Application No. PTC/US2005/032392, the disclosure of which is incorporated by reference in its entirety.

A design file containing the desired microarray positions of the DATAS expression vectors was uploaded into a high resolution scanner. The expression vectors containing the DATAS fragments were mixed with gelatin and lipofectamine and then spotted in duplicate in a pattern that matched the design file uploaded into the high resolution microarray scanner. The following positive controls, negative controls and experimental samples were spotted on the array.

a) Vector with no insert (negative control)

b) Vector encoding enhanced green fluorescent protein (EGFP)

c) Vectors containing DATAS fragment inserts

d) Vector containing influenza NP encoding cDNA (positive control)

The spotted arrays were then overlaid with HLA class I (e.g. HLA-A2) expressing adherent 293T cells and cultured for 48-72 hours to allow cells time to express vector-encoded transcripts.

The assay for CTL reactivity was then performed by incubating HLA-matched CD8⁺ T cells from normal and prostate cancer patients on cultured reverse transfected microarray slides for 4 hours. Prior to incubation with the array, T cells from normals or patients may need to be expanded in vitro, to a small or large extent, to enhance the frequency of responding T cells. An inhibitor of activated caspase, fluorescence-tagged (sulfo-Rhodamine)-Z-VAD, is present throughout the cell incubation to bind caspases that become activated when antigen expressing adherent cells on certain spots are induced to die by the CTL. The slides are then washed to remove unbound fluorescent Z-VAD and 2-color (Green and Red) fluorescence images of the microarray slides are obtained with the microarray scanner.

Analysis and confirmation of data is then determined by visual inspection of the scanned image. The success of the reverse transfection is evaluated by observing green fluorescence at EGFP vector control spots. Then the fluorescence threshold(s) are adjusted on microarray scanner as necessary to establish relevant background and positive control signal intensities. Next, DATAS spots where T cells have had a positive response are identified by the presence of a red fluorescent signal. Using the coordinate locations for the spots, the cDNAs yielding positive responses are identified.

### Expression Monitoring:

A valid epitope target for prostate cancer requires that the expression of the epitope be limited to prostate tissue, or preferably to prostate tumors. Assessment of the expression profile for each prioritized sequence was performed by RT-PCR, a procedure well known in the art. A protocol known as touchdown PCR was used, described in the user's manual for the GeneAmp PCR system 9700, Applied Biosystems. Briefly, PCR primers were designed to the DATAS fragment and used for end point RT-PCR analysis. Each RT reaction contained 5 μg of total RNA and was performed in a 100 μl volume using Archive RT Kit (Applied Biosystems). The RT reactions were diluted 1:50 with water and 4 μl of the diluted stock was used in a 50 μl PCR reaction consisting of one cycle at 94° C. for 3 min, 5 cycles at 94° C. for 30 seconds, 60° C. for 30 seconds and 72° C. for 45 seconds, with each cycle reducing the annealing temperature by 0.5 degree. This was followed by 30 cycles at 94° C. for 30 seconds, 55° C. for 30 seconds, and 72° C. for 45 seconds. 15 μl was removed from each reaction for analysis and the reactions were allowed to proceed for an additional 10 cycles. This produced reactions for analysis at 30 and 40 cycles, and allowed the detection of differences in expression where the 40 cycle reactions had saturated. The level of expression profile of the DATAS fragment was determined in normal and tumor prostate total RNA, as well as total RNA from normal samples of brain, heart, liver, lung, kidney, colon, bone marrow, muscle, spleen, and testis. Expression profiles were prioritized accordingly for specific expression in prostate tumor and low expression found in normal tissues, including normal prostate.

### Verification of RNA Structure:

DATAS identifies sequences that are altered between the experimental samples. However, the exact sequence of the junctions or borders that the DATAS fragment represents can not be determined directly from the isolated DATAS fragment sequence. The DATAS fragment was used, however, to design experiments that elucidate the sequence of each transcript present in each sample. Primers were designed to amplify a region of the gene larger than the proposed DATAS fragment sequence. These amplicons were subsequently cloned and sequenced for the identification of the exact junctions of all exons and introns. This required partial cloning of the isoforms from an identified sample to verify the primary structure (sequence) of the isoforms. All twenty samples (10 normal and 10 tumor samples) initially used to generate the DATAS libraries were used for the verification of the mRNA structure of the prioritized genes.

### Isolation of Full-Length Clones of Isoforms:

Isolation of the full-length clones containing both isoforms was accomplished utilizing the information and DNA fragments generated during the structure validation process. Several methods are applicable to isolation of the full length clone. Where full sequence information regarding the coding sequence is available, gene specific primers were designed from the sequence and used to amplify the coding sequence directly from the total RNA of the tissue samples. An RT-PCR reaction was set up using these gene specific primers. The RT reaction was performed as described infra, using oligo dT to prime for cDNA. Second strand was produced by standard methods to produce double stranded cDNA. PCR amplification of the gene was accomplished using gene specific primers. PCR consisted of 30 cycles at 94° C. for 30 seconds, 55° C. for 30 seconds, and 72° C. for 45 seconds. The reaction products were analyzed on 1% agarose gels and the amplicons were ligated into prepared vectors with A overhangs for amplicon cloning. 1 μl of the ligation mixture was used to transform E. coli for cloning and isolation of the amplicon. Once purified, the plasmid containing the amplicon was sequenced on an ABI 3100 automated sequencer.

Where limited sequence information was available, the oligo pulling method was utilized. Briefly, a gene-specific oligonucleotide was designed based on the DATAS fragment. The oligonucleotide was labeled with biotin and used to hybridize with a single stranded plasmid DNA library prepared from either normal prostate tissue or prostate tumor tissue following the procedures of Sambrook et al (1989). The hybridized cDNA was separated by streptavidin conjugated beads and eluted by heating. The eluted cDNA was converted to double strand plasmid DNA and used to transform E. coli cells and the longest cDNA clone was subjected to DNA sequencing.

### Results

Using methods described above, 23 DNA fragments have been identified that putatively correspond to exons (novel splice variants) expressed by prostate tumor tissue and elicit a productive CTL response. Sequence Ids and corresponding patient IDs that yielded positive results are summarized in Table 1. Repeat responders and the type of splice event represented by the DATAS fragment clone are also listed in Table 1.

These DNA sequences are contained in Tables 2 and correspond to the nucleic acid sequences having SEQ ID NOS: 1-23. Genomic sequence locations were generated using BLAT and the UCSC Genome Browser referencing the March 2006 human genomic assembly.

A predictive algorithm was used to identify potential HLA-A2 binding peptides that may correspond to the antigenic peptides contained in each DATAS fragment clone that stimulated the CLT response monitored in the described CTL assay. The results of this analysis are contained in Table 3. Peptides are listed for each possible frame encoded by the fragment cloned into the expression vector. On the left of each predicted peptide epitope is a number that indicated the location of the N-terminal amino acid of that peptide in the parent protein listed to the left. On the right of each peptide is a score from 1 to 30, which indicates the likelihood of higher affinity binding to HLA-A2 as the number increases towards 30.

TABLE 1
CTL assay positive hits

| Patent | Patient ID | Repeat | |
|---|---|---|---|
| Sequence | with | positve | |
| ID | positive hit | hits | event type |
| No. 1 | EYGRT | | exon extension |
| No. 2 | EYGRT; MSMPB | | exon extension |
| No. 3 | MSMPB | | exon extension |
| No. 4 | P72 | | exon extension |
| No. 5 | EYGRT | | exon extension |
| No. 6 | EYGRT | | exon extension |
| No. 7 | P72 | P72 | exon extension |
| No. 8 | MSMPB | | exon extension |
| No. 9 | EYGRT | | novel exon |
| No. 10 | MSMPB | | novel exon |
| No. 11 | MSMPB; P72 | | novel exon |
| No. 12 | P72 | | novel exon |
| No. 13 | MSMPB | | exon extension |
| No. 14 | MSMPB | | exon extension |
| No. 15 | MSMPB | | genomic hit |
| No. 16 | P72 | P72 | exon extension |
| No. 17 | P72 | P72 | genomic hit |
| No. 18 | FRJY5 | | exon extension |
| No. 19 | EYGRT | | novel exon |
| No. 20 | P72 | | exon extension |
| No. 21 | EYGRT | | exon extension |
| No. 22 | P72 | | exon extension |
| No. 23 | P72 | P72 | genomic hit |

TABLE 2
Sequence information of the DATAS fragments

EntrezGene ID: 51016
Genomic sequence: chr 14: 23, 680, 141-23, 680, 474
Sequence definition: chromosome 14 open reading frame 122
Verified sequence:
Sequence ID: No. 1
CTACCGGGGGAAGGGTCAAAGAGCGTGCCGAAGCGCTGGAGGGAGCTTCACGGACGCGAGCTAGGCACCGGCT
CGCCTAATCCGGTACTAATCCGGCTTGCTGCTTCCCGTCCAGGCCTCGCTCGCCATGGGGGAGGTGGAGATCT
CGGCCCTGGCCTACGTGAAGATGTGCCTGCATGCTGCCCGGTACCCACACGCCGCAGTCAACGGGCTGTTTTT
GGCGCCAGCGCCGCGGTCTGGAGAATGCCTGTGCCTCACCGACTGTGTGCCCCTCTTCCACAGCCACCTGGAC
CTGTCCGTCATGTTGGAGGTCGCCCTCAACCAGGTGCCTCCGTTGCAT
EntrezGene ID: 84939
Genomic sequence: chr19: 1, 327, 655-1, 328, 088
Sequence definition: melanoma assoc. antigen (mutated) 1
Verified sequence:
Sequence ID: No. 2
GGCTCAGGGGGCACGTTTGCGTTTGGACCTGTCTGCGCGTTCTCCTGCGTGGCAGTCCTGATTTCCATACTTC
TGGAGAATCCATTTCGTTAACACTGAAAGCCAGTTCTCTTTTCCTGGCAGTTTTTTTCATTTTATTTTTGGCA
TTTTTTACAAGATACCGTTCGGGAAAGGCTTTTGAAAGGACGGAAGCGTATTCACTGTGCGCCAGTACTCCTG
GCTGTGCTGTGGTTTCTCCCGACGTGCACATCGATCTCGTATGTGTGGCATCTGATATTAAACGGGAGGTTTT
AAGAAGCGTCTGCCGTGATCATGGAGCTTCGGAAGCGGGAATGGTTCTTCCGGGTTTGCTGTTTTGTCTGTTT
CCCCCTTGTGTGGTTTCCGCCTGCGACAGTTCCAGAATTTGCTCTCCCACTCAGTGTGCTCTGCAGCTG
EntrezGene ID: 95
Genomic sequence: chr3: 51, 996, 641-51, 996, 968
Sequence definition: aminoacylase 1
Verified sequence:
Sequence ID: No. 3
CCAGGCAGCTGGCGAGGGGGTCACCCTAGAGTTTGCTCAGGTATGGACTTGGGACATGTGATGGGAGAGTGTG
GGAGCCGGGGGAGACCCAAGTGTGCAACAGTGGAGTGTGTGCTTGGTGTGTCTGCATATGTCTGGGCATTTCT
TTATCTGTGACAGACACATTTTATTCCAACAAGCATTCATTGTAGAGGCCACTGTGGGTGCTGGGGAATGCTG
TGGGGAGTAAAATTAGGCACAGTTCATGCCCTTGTATGGTGAAACGGGGAGATATAAATCAAACATTTATGTG
ATATTACTTTTTTCTGAGAGAATCTCACTCCGTCAC
EntrezGene ID: 5110
Genomic sequence: chr6: 150, 159, 247-150, 165, 314
Sequence definition: protein-L-isoaspartate (D-aspartate)
O-methyltransferase (PCMT1)
Verified sequence:
Sequence ID: No. 4
GTAAGGGATGGAAGAATGGGATATGCTGAAGAAGCCCCTTATGATGCCATTCATGTGGGAGCTGCAGCCCCTG
TTGTACCCCAGGCGCTAATAGATCAGTTAAAGCCCGGAGGAAGATTGATATTGCCTGTTGGTCCTGCAGGCGG
AAACCAAATGTTGGAGGAGTATGACAAGCTACAAGATGGCAGCATCAAAATGAAGCCTCTGATGGGGGTGATA
TACGTGCCTTTAACAGATAAAGAAAAGCAGTGGTCCAGGTGGAAGTGATTTTATCTTCTGCTCTTTCTTCTTC
CACACATGCAAGGTGAAAGGGTGTGGATTTTAAGACATTAGACTACAAGAGCTGTTTTTGGTTGTCACCTTTA
TGCTCCTCAGA
EntrezGene ID: 24142
Genomic sequence: chr3: 50, 309, 814-50, 311, 728
Sequence definition: N-acetyltransferase 6
Verified sequence:
Sequence ID: No. 5
GTAAAGGCATGTGGAATCCAGGGGCAGCTTTGGCTGGCACGCAGGATCCAGAGTCAGCTCAGCTGGGCTGGTA
CTCAGGATCAGCTCCATCCGGTGTGTAGGGTCTAGTGTAGGGGTCAGCTTGGCTGGGCCAGGGCTCAGAGTCA
GCTCTTGCCTATGCACAGGATCCAGGTTCAGCTGAGTCAGGCTGGGAGCCAAGGTCACCTGCTGCTAGGTTGC
AGGTGGCTCAGTGCCAGGCTGGAGGTTAAGACCCCAGTCTCCAGGCAGTAGCATCTCTTCAGACCACAGTGGC
TCTCCTCATGTTGATGCTGGCCTCTGGGATGTTCCGCGTCCTAGCTCCGCACAGCTGGGTATCTCACTCAGTC
GCCACCTCGGCCTCCCAACA
EntrezGene ID: 7113
Genomic sequence: chr21: 41, 762, 326-41, 762, 782
Sequence definition: transmembrane protease, serine 2
Verified sequence:
Sequence ID: No. 6
GCCGAGGTCTTCCCTAAGGACAGGGAGACTTGTTGAGCTCCCAGTGTTGCCTGCAGCTCTCGCAGGGAGAGCA
CACTTCCCTCCCTGAGACCTCCACACGCACTACACAGATGGTCACAGCCCTATTCTCCAGCCCTGCCCCCGTC
AGCTCATCCCAGCACCAACCCCTCTCCTGGGAAACCTGGATTCTCCTTACAACTGGCGGTGCCTGTGCTGAAT
GCAGCTCTGGAGTTCAGCTTCAGGGAAAGAAATTCCCAGCTCTGAAACTATAGGGCTCTATCAGTGCCATGGA
ATCCCCCTCTTATTCACCCAACGGCTTGGTTCTGCTGAACCAATTTCCCACTCCTCACTGAACCCCACCCAGG
ACACACAGGCGCCTGTTTCCCCTCTGGCATGCACACACTCTTCTGGAGCACACGCGACCCTCCCTAGGCCGCC
TGCTTCTCGGCTCCCCTGCACA
EntrezGene ID: N/A
Genomic sequence: chr10: 76, 837, 410-76, 837, 825
Sequence definition: GenBank Human mRNA BC029963
Verified sequence:
Sequence ID: No. 7
CCGATCGGGGAAGTTTGCAGGGCAGGCGGCGGCAAGTTATTTAAGCACTGGCTCGAAGTGAGGACAGAGAACT
GGGCAGGAGGTTACCCAGGCCAATTCTTGGGCCATTCCGCCCCAAGGAACATGGGAGCTGTCCCCAAGCTTTC
TTGAGCCCCGCCGGGCGGACGCTTCTCGGCTAATCTTTCTGCAACTTTTACAAAGCTGCCTCCTCCCCAGTTT
GATCTCTGCAAACACATCCTGGGCGTCCGCGGCAGATTGGAAACTATTTTTACCGTCTGGGGCCGAGGAGGTG
CATTGAAATAAGGAAGGCGAGGCGCCTTCTCTAGCGTTTCACCCCGGACCAGGCTGGGTCACCCAGTGGTCTC
TTTGCTCCCCCGCCCCACCCTTCGTGCGCCTTCCAGAGCCGCCATCGAATCTCTCTTGC
EntrezGene ID: 11273
Genomic sequence: chr16: 28, 750, 682-28, 751, 068
Sequence definition: ataxin 2-like
Verified sequence:
Sequence ID: No. 8
GTAGCAGGGCAGCAGACTTGGGAGAACGCGGGGGATACATCCGGCCCACTGGAAGAAAAGGGGGAGCTGTAGG
ATGGTTACTGCTGGTCAACAGCAGTGAGTACAAGGGCCTTACTCAAAACAGCAAGAAAGTTTACACACTATAC
AACCAGAGTCACCAACTTAAATGTCTAGAAGCTCAAGGCAGGTAACACAAGCAGGTAAATGGCCTCATGGGCA
TCATCGACAACTGGAATGCTTAAAGGAGTGGCAGGGATTCTGTTCCAGTCAACTCAAGCTACGCAGCAAGGCA
AGCTCAGTTGACAGAATTTCCTGTAATTCAAAAGAAACTAAAAATTTTCTATGGCTTCTATTGATTTATTTAT
TTAGAGACAGGGTATCGCTCTGTTCCCC
EntrezGene ID: 5590
Genomic sequence: chr1: 2, 047, 301-2, 047, 449
Sequence definition: protein kinase C, zeta
Verified sequence:
Sequence ID: No. 9
GGGCAAAGGATGTTGCCCCTGTCCTGAAGGCTGTCGCCCGATCGCTCTATCCAAGGCTGCCCTGGGGCAGCGT
CACCTGGGGGTCCTGCGGGGGCTTCTCAGCACAGCATCCAGCACTGCCACCTAGTGTGTTCCCGTCACGTCTC
CTCCCCCCG
EntrezGene ID: 7037
Genomic sequence: chr3: 197, 286, 590-197, 293, 338
Sequence definition: transferrin receptor (p90, CD71)
Verified sequence:
Sequence ID: No. 10
GTGGCGAGGCGGTTCGGGACGGAGGACGCGCTAGTGTTCTTCTGTGTGGCAGTTCAGAATGATGGATCAAGCT
AGATCAGCATTCTCTAACTTGTTTGGTGGAGAACCATTGTCATATACCCGGTTCAGCCTGCTCTCC
EntrezGene ID: 64072
Genomic sequence: chr10: 73, 173, 842-73, 174, 176
Sequence definition: cadherin-like 23
Verified sequence:
Sequence ID: No. 11
GACACCCTAGCTGCAAGGGAGGTTAGAAAAAATGAGGGACAGGATTATCATGGTCAGCATATTACTTGGGGGC
CAGATATATCACTTCCCACACAAAAGCAGGCCTCTGTCAGTGAGGAAGAAGTGGAGAATGGATTTGGGGTGGG
CATCTGACAGCATTTGCCACACTGCCCTCCCCAATCCTAATGCAGGCCAGTCAACGCACCACCCCCACTCCCC
ACCCACACTCCAACCCCCACAGTCCTAGGGTCTCTCAGGGAGACCATCGGTGGAATCCATAACATTCTAAGAC
CCTGCAGTTTGTAGGCAAAATCAGGCTTCCTTTGGGTGCCCCCG
EntrezGene ID: 65217
Genomic sequence: chr10: 56, 047, 284-56, 047, 451
Sequence definition: Homo sapiens protocadherin 15 (PCDH15)
Verified sequence:
Sequence ID: No. 12
GTTGAGACCTCGAATTACAGGCATGAGCCACCGTGCCTGGCCTTTTCTTTTCTTTTAAGCTACTTTTTAATAT
ATAGTAATGACTGTTAATATAGTATATACTATGCTATTCATCAATGCTGTAACTTTCTTAGTTTCATTTTCTC
ACTCAATTGAAGTCCAGGTACCCAGGTCCACACG
EntrezGene ID: 57194
Genomic sequence: chr15: 23, 479, 621-23, 484, 596
Sequence definition: ATPase, Class V, type 10A
Verified sequence:
Sequence ID: No. 13
GGAGGAGGTCGGCATCACCAGTAACAGGAATCCCTCTCCCTTTGAAGGCAGTGATGGCCAGCGACTTTGCAGT
GCCGAAATTCCGATACCTGGAGAGGCTCTTGATTCTTCACGGGCATTGGTGCTACTCCCGACTTGCCAACATG
GTGCTGTACTTCTTCTACAAAAACACAATGTTCGTGGGCCTCCTGTTTTGGTTCCAGTTTTTCTGTGGCTTCT
CTGCATCTACCATGATTGACCAGTGGTATCTAATCTTCTTTAATCTGCTCTTCTCGTCACCTCCAACGA
EntrezGene ID: 7701
Genomic sequence: chr2: 219, 231, 947-219, 232, 130
Sequence definition: zinc finger protein 142 (clone pHZ-49)
Verified sequence:
Sequence ID: No. 14
CGGACCAGGACAAAATCATGACGATGCTCCATCCCAAGCCTTCCAGGTTACAAACCGCCTTTCCAGTTCGCTG
GCTCTTATGATCCAGCTCCTGCCCGACGATGTGGACACTGTCATCACCTCCATTTTACAGATAGGAAAGCTGA
GGCCCAGAGAAGCGAAGCGACTGTGTCTGTCCAAGACCACGCGCCCTCCTCCCTGTC
EntrezGene ID: N/A
Genomic sequence:
Sequence definition: GenBank Human mRNA AF116698 (intronic hit)
Verified sequence:
Sequence ID: No. 15
CGGCGCACCTGGTACGTGGTGTATGTTCCATTTGTGCTGGCCACGGTGATGATGATGATGATGCCATCTCTCC
GGCTACACTGCGAGCACAGATCACAGCTCTCTGTAGCTGATGCTGGCTCTACCCACTGTATTCGGG
EntrezGene ID: 26993
Genomic sequence: chr19: 15, 372, 085-15, 372, 898
Sequence definition: A kinase (PRKA) anchor protein 8-like, AKAP8L
Verified sequence:
Sequence ID: No. 16
GGGGAGGCACCCCAGGAAAACTCACCCTTCTCTGGATCCTCTTTGCCTTCTTCTCTCCCATCCTCTTTTCCCT
CCTCATCCTCTCCGTCCACTCTGGAGCCCTTCTCCACAGCCTCGCACCC
EntrezGene ID: N/A
Genomic sequence: chr4: 171, 259, 069-171, 259, 517
Sequence definition: genomic hit
Verified sequence:
Sequence ID: No. 17
GCCGCGGGATTGAAACGTTATACAGCGGACCCAGTGAGACACCAGCTGGGGTGGCCAAAGGAGTGCTAGTGTC
ACCCCTCTCCTAACTCCAGAAAGCACAGCTTGCAGCTCTAGGAGAGACTCCTTTTGGTTGAGGAAAGGAAAGG
GAAGAGTAAAGAGGATTTTGTCTGGCAACTGGGATACCAGCCCAGCCACAGTAACATAAAACAACAAGCAGAT
CCCTGAAGTCTCCATTCCAGGCCCTAGCTCCTGGATAACATTTCTAGACCCACCTTGGGCCAGAAAAGAACCT
GTTACCCTGAAGGAAAAAACAAAGTCCTGGAAGAACTCACCACCTGCTGACTAAAGAGCCCGTAGTCCTTGAA
TAAACATTAGTAGCAGCCAGGCAGTACTCATCACAGACCTAGAACAGTGACGGCCATGGGAAAAGACTCTTTT
TGAAGAAAGGAGAGGGAGGAGTAAAAAGGACTTTGTCT
EntrezGene ID: 9570
Genomic sequence: chr17: 42, 371, 388-42, 371, 614
Sequence definition: golgi SNAP receptor complex member 2
Verified sequence:
Sequence ID: No. 18
GGCCTTAGGGGCACGAGGATGTGAATCGACCTACTACTTAATATAATGGCTGTGAGAAAAGGCCTCTTTCCTT
TCCTTTCCACTTTTGCTCCACCCTATCAGGAGCCAGAAAGGCCTGATGGTGACAAGGTGTGAACCGTGTGGAC
AGTCCTGCACACAGGGCTCACTGTGGACACCTTCCCAGGGCACGCATCAGCTGTGTGAACAAAGCCACCAAAG
CCATTCTTTCCCTCCCCCGC
EntrezGene ID: N/A
Genomic sequence: chr13: 21, 727, 184-21, 727, 549
Sequence definition: GenBank human mRNA AK054845
Verified sequence:
Sequence ID: No. 19
GGGGACAGGCAGAGGTTGCACTGCACTCCAGCCTGGGTGACAGATCAAGATTCTGTCTCAAGTAATAATAATG
ATGATGATGATGATGATGATGATGATGATAATAACACACCTTGTTAATGTTAATTCACTGTGTTAAATCAATT
AACTCATTTTCTCATAATTACCCTTGAACGAGTAAGTTATTGACATTCTCTCCATGGGAAAAAAAACCCTTGG
AGAAAGGTAACTTGAATATGGCCAAATGGGCAATTTGGATAGAACTGGGATTTGAACCTAGGACATCTGATTC
CACATCCTTTTCATGAGCCACAATGCTCTACATGTGGTTTTATTTCTTGGATCACATGTAACACATCCATAAA
AGTGCCTCCCCCCG
EntrezGene ID: 29091
Genomic sequence: chr14: 24, 351, 679-24, 351, 865
Sequence definition: Homo sapiens syntaxin binding protein 6 (amisyn)
(STXBP6)
Verified sequence:
Sequence ID: No. 20
CTCCCGGCCGCCATGGCCGCGGGATGTCCCGAATTCTTGTAAAACTGCTGAACAAACTCTCCAGTTTCTCTTA
AGAAGAGTCACCAGGATAGGCAGTTTCTCAACATTTGTGCTTCATGGCAAGCTAAGGAGAGAGAGGAATGAGA
AAAGTAAGACTTTATTAGCAGGTATTAGCAGAGAGATTTCACTGTGCTGCTCCCTCTCCACCTCCCCC
EntrezGene ID: N/A
Genomic sequence: chr6: 146, 238, 220-146, 238, 476
Sequence definition: GenBank human mRNA AK123820
Verified sequence:
Sequence ID: No. 21
GGCGGCAGGTCTAATCTGGCACACTTGCTAACTTCTAATAAAGAATCAAATGGTCTTCCAACTCACATAATGA
CTAATTCTAAAGCATGCAACCTCAAAGTACTACATTCTCCCTGTTCTAGAAATACAACCCTAGAGGGCCAGTC
TTCCTTCAGCATTCAAATATTGAGTAGAACAACTGAACTGTAAGGTTCAACTAAAGGAATGCAACTGTGACTT
GAGCTGTGGAACGGCTATGGCCTAAGTCAAGAATTCTCAACCTCCCAC
EntrezGene ID: 4343
Genomic sequence: chr1: 113, 044, 178-113, 044, 827
Sequence definition: Homo sapiens Moloney leukemia virus 10, (MOV10) - ID
Verified sequence:
Sequence ID: No. 22
GTTAGGAGGTAGAGGGGCTGAGCTTGCTCAGACCTTGCAGTAAATTCTGTCCCTGTTGCAGGTCCAGTTTGGC
AGGGAAGGGACACCCGGTATACCCTCCGTTTTCTTTACAGAACTCCAGGAATCTGTGGGGTACAGAGGAGTGC
CAGCAGAGACTGGAGGCTAAGCCACGGTCCTGTCCCATCTGAGCTGTACTTGCTCAACCTCTGGATGTCATTT
AACTTATAAATACAATAGTGATGCTGTGAAAATGGACACATCTGCGGATTAACTGAGCGATAAGCAAGCGCTT
AAAAAAGATCCACCTGCCCCCA
EntrezGene ID: N/A
Genomic sequence: chr15: 78, 115, 339-78, 115, 450
Sequence definition: genomic hit
Verified sequence:
Sequence ID: No. 23
GCCCCCACCTGCTGCCCAGTTCCTCTATGATCCAGCTCTAATGCTTCCCAAATTATAAGGTGAATAAAAATCC
TTAAGGACCTTGTCAAAATGACAACACTGATTTAGTAGGTCCGCACG

TABLE 3
HLA-A2 binding peptide analysis

| | Amino acid sequence | HLA-A2 peptide 1 | HLA-A2 peptide 2 | HLA-A2 peptide 3 |
|---|---|---|---|---|
| No. 1 | | | | |
| Frame 1 | LPGEGSKSVPKRWRELHGRELGTGSPNPVLIR | (32)RLAASRPGL(24) | (71)AVFGASAAV(22) | (48)DLGPGLREDV(21) |
| | LAARPGLARHGGGGDLGPGLREDVPACCPVPT | | | |
| | RRSQRAVFGASAAVWRMPVPHRLCAPLPQPPG | | | |
| | PVRHVGGRPQPGASVA | | | |
| Frame 2 | YRGKGQRACRSAGGSFTDAS | (11)SAGGSFTDA(12) | (10)RSAGGSFTD(8) | (4)KGQRACRSA(7) |
| Frame 3 | TGGRVKERAEALEGASRTRARHRLA | (11)ALEGASRTRA(17) | (4)RVKERAEAL(16) | (15)ASRTRARHRL(13) |
| No. 2 | | | | |
| Frame 1 | GSGGTFAFGPVCAFSCVAVLISILLENPFR | (12)CAFSCVAVL(21) | (16)CVAVLISIL(21) | (19)VLISILLEN(20) |
| Frame 2 | AQGARLRLDLSARSFAWQS | (5)RLRLDLSAR(15) | (2)QGARLRLDL(14) | (7)RLDLSARSPA(14) |
| Frame 3 | LRGHVCVWTCLRVLLRGSPDFHTSGESISLTL | (112)VLPGLLFCL(28) | (38)FLAVFFILFL(28) | (36)SLFLAVFFI(25) |
| | KASSLFLAVFFILFLAFFTRYRSGKAFERTEA | | | |
| | YSLCASTPGCAVVSPDVHIDLVCVASDIKREV | | | |
| | LRSVCRDHGASEAGMVLPGLLFCLFPPCVVSA | | | |
| | CDSSRICSPTQCALQL | | | |
| No. 3 | | | | |
| Frame 1 | PGSWRGGHPRVCSGMDLGHVMGECGSRGRPKC | (33)ATVECVLGV(24) | (37)CVLGVSAYV(22) | (3)SWRGGHPRV(17) |
| | ATVECVLGVSAYVWAFLYL | | | |
| Frame 2 | QAAGEGVTLEFAQVWTWDM | (22)QAAGEGVTL(1) | (6)GVTLEFAQV(16) | (2)AAGEGVTLE(14) |
| Frame 3 | RCLARGSP | no data | no data | no data |
| No. 4 | | | | |
| Frame 1 | VRDGRMGYAEEAPYDAIHVGAAAPVVPQALID | (51)QMLEQYDKL(24) | (63)SIKMKPLMGV(24) | (69)LMGVIYVPL(22) |
| | QLKPGGRLILPVGPAGGNQMLEQYDKLQDGSI | | | |
| | KMKPLMGVIYVRLTDKEKQWSRWK | | | |
| Frame 2 | no data | no data | no data | no data |
| Frame 3 | KGWKNGIC | no data | no data | no data |
| No. 5 | | | | |
| Frame 1 | CWEAEVATE | (1)CWEAEVATE(7) | (2)WEAEVATE(6) | (1)CWEAEVATE(1) |
| Frame 2 | VGRPRWRLSEIPSCAELGRGTSQRPAST | (9)SEIPSCAEL(20) | (7)RLSEIPSCA(16) | (2)GRPRWRLSEI(15) |
| Frame 3 | LGGRGGD | no data | no data | no data |
| No. 6 | | | | |
| Frame 1 | CAGEPRSRRPREGRVCSRRVCACQRGNRRLCV | (28)RRLCVLGGV(17) | (25)RGNRRLCVL(15) | (7)SRRPREGRV(14) |
| | LGGVQ | | | |
| Frame 2 | VQGSREAGGLGRVACAPEECVHARGETGACVS | (5)REAGGLGRV(20) | (13)VACAPEECV(16) | (23)ARGETGACV(16) |
| | WVGFSEEWEIGSAEPSRWVNKRGIPWH | | | |
| Frame 3 | CRGAEKQAA | (2)CRGAEKQAA(5) | no data | no data |
| No. 7 | | | | |
| Frame 1 | AREIRWRLWKAHEGWGGGAKRPLGDPAWSGVKR | (22)PLGDPAWSGV(19) | (3)EIRWRLWKA(15) | (23)LGDPAWSGV(15) |
| Frame 2 | QERFRDGGSGRRTKGGAGEQRDHWVTQPGPG | (15)GAGEQRDHWV(18) | (16)AGEQRDHWV(15) | (11)RTKGGAGEQ(9) |
| Frame 3 | KRDSMAALEGARRVGRGSKETTG | (6)AALEGARRV(22) | (5)MAALEGARRV(16) | (7)ALEGARRVGR(16) |
| No. 8 | | | | |
| Frame 1 | GEQSDTLSLNK | (1)GEQSDTLSL(16) | (1)GEQSDTLSLN(5) | (2)EQSDTLSLN(2) |
| Frame 2 | GNRAIPCL | | no data | no data |
| Frame 3 | GTERYPVSK | GTERYPVSK(9) | no data | no data |
| No. 9 | | | | |
| Frame 1 | GQRMLPLS | no data | no data | no data |
| Frame 2 | GKGCCPCPEGCRPIALSKAALGQRHLGVLRGL | (20)ALGQRHLGVL(26) | (28)VLRGLLSTA(24) | (19)AALGQRHLGV(22) |
| | LSTASSTAT | | | |
| Frame 3 | AKDVAPVLKAVARSLYPRLPWGSVTWGSCGGF | (7)VLKAVARSL(22) | (3)DVAPVLKAV(21) | (14)SLYPRLPWG(20) |
| | SAQHPALPPSVFPSRLLPP | | | |
| No. 10 | | | | |
| Frame 1 | VARRFGTEDALVFFCVAVQNDGSS | (10)ALVFFCVAV(25) | (9)DALVFFCVAV(18) | (3)RRFGTEDALV(15) |
| Frame 2 | WRGGSGRRTR | (1)WRGGSGRRT(6) | (2)RGGSGRRTR(4) | (1)WRGGSGRRTR(4) |
| Frame 3 | GEAVRDGGRASVLLCGSSE | (3)AVRDGGRASV(21) | (4)VRDGGRASV(19) | (5)RDGGRASVLL(14) |
| No. 11 | | | | |
| Frame 1 | DTLAAREVRKNEGQDYHGQHITWGPDISLPTQ | (28)SLPTQKQASV(26) | (21)ITWGPDISL(22) | (20)HITWGPDISL(19) |
| | KQASVSEEEVENGFGVGI | | | |
| Frame 2 | TP | no data | no data | no data |
| Frame 3 | HPSCKGG | no data | no data | no data |
| No. 12 | | | | |
| Frame 1 | VETSNYRHEPPCLAFSFLLSYFLIYSNDC | (12)CLAFSFLLSY(18) | (18)LLSYFLIYSN(18) | (17)FLLSYFLIYS(17) |
| Frame 2 | LRPRITGMSHRAWPRLFF | (7)GMSHRAWPFL(20) | (4)RITGMSHRA(14) | (8)MSHRAWPFL(11) |
| Frame 3 | no data | no data | no data | no data |
| No. 13 | | | | |
| Frame 1 | GGGRHHQ | no data | no data | no data |
| Frame 2 | EEVGITSNRNPSPFEGSDGQRLCSAEIPIPGE | (64)VLVPVFLWLL(27) | (33)ALDSSRALV(24) | (51)VLLLQKHNV(24) |
| | ALDSSRALVLLPTCQHGAVLLLQKHNVRGPPV | | | |
| | LVPVFLWLLCIYHD | | | |
| Frame 3 | RRSASPVTGIPLPLKAVMASDFAVPKFRYLER | (9)GIPLPLKAV(24) | (76)TMIDQWYLI(20) | (28)RYLERLLIL(19) |
| | LLILHGHWCYSRLANMVLYFFYKNTMFVGLLF | | | |
| | WFQFFCGFSASTMIDQWYLIFFNLLFSSPPT | | | |
| No. 14 | | | | |
| Frame 1 | RTRTKS | no data | no data | no data |
| Frame 2 | GPGQNHDDAPSQAFQVTNRLSSSLALMQLLPD | (29)QLLPDDVDTV(30) | (30)LLPDDVDTV(29) | (23)SLALMIQLL(28) |
| | DVDTVITSILQIGKLRPREAKRLCLSKTTRPP | | | |
| | PC | | | |
| Frame 3 | DQDKIMTMLHPKPSRLQTAFPVRWLL | (8)MLHPKPSRL(23) | (17)QTAFPVRWL(20) | (7)TMLHPKPSRL(20) |
| No. 15 | | | | |
| Frame 1 | RRTWYVVYVPFVLATVMMMMMPSLRLHCEHRS | (8)YVPFVLATV((23) | (16)VMMMMMPSL(23) | (18)MMMMPSLRL(21) |
| | QLSVADAGSTHCIR | | | |
| Frame 2 | GAPGTWCMFHLCWPR | (2)APGTWCMFHL(12) | (7)CMFHLCWPR(10) | (1)GAPGTWCMF(7) |
| Frame 3 | AHLVRGVCSICAGHGDLCS | (2)HLVRGVCSI(26) | (9)SICAGHGDL(22) | (1)AHLVRGVCSI(18) |
| No. 16 | | | | |
| Frame 1 | GCEAVEKGSRVDGEDEEGKEDGREEGKEDPEK | (3)EAVEKGSRV(14) | (2)CEAVEKGSRV(12) | (10)RVDGEDEEG(8) |
| | GEFSWGAS | | | |
| Frame 2 | GARLWRRAPEWTERMRREKRMGEKKAKRIQRR | (20)RMGEKKAKRI(17) | (3)RLWRRAPEWT(15) | (29)IQRRVSFPGV(14) |
| | VSFPGVPP | | | |
| Frame 3 | VRGCGEGLQSGRRG | (3)GCGEGLQSG(11) | (2)RGCGEGLQSG(8) | (1)VRGCGEGLQ(3) |
| No. 17 | | | | |
| Frame 1 | RQSPFYSSLSFLQKESFPMAVTVLGL | (18)PMAVTVLGL(23) | (17)FPMAVTVLGL(18) | (16)SFPMAVTVL(16) |
| Frame 2 | DKVLFTPPSPFFKKSLFPWPSLF | (14)KSLFPWPSL(16) | (15)SLFPWPSLF(15) | (3)VLFTPPSPF(13) |
| Frame 3 | TKSFLLLFLLSSKRVFSHGRHCSRSVMSTAWL | (32)LLLMFIQGL(30) | (31)WLLLMFIQGL(27) | (34)LMFIQGLRAL(26) |
| | LLMFIQGLRAL | | | |
| No. 18 | | | | |
| Frame 1 | AGEGKNGFGGFVHTADACPGKVSTVSPVCRTV | (17)ACPGKVSTV(21) | (30)RTVHTVHTL(21) | (16)DACPGKVSTV(21) |
| | HTVHTLSPSGLSGS | | | |
| Frame 2 | RGRERMALVALFTQLMRALGRCPQ | (10)ALFTQLMRAL(24) | (7)ALVALFTQL(23) | (3)RERMALVAL(17) |
| Frame 3 | GGGKEWLWWLCSHS | (2)GGKEWLWWL(16) | (1)GGGKEWLWWL(15) | (6)WLWWLCSHS(12) |
| No. 19 | | | | |
| Frame 1 | RGEALLWMCYM | (3)EALLWMCYM(7) | (2)GEALLWMCYM(7) | (1)RGEALLWMC(4) |
| Frame 2 | GGRHFYGCVTCDPRNKTTCRALWLMKRMWNCM | (23)WLMKRMWNQM(16) | (15)KTTCRALWL(16) | (9)VTCDPRNKT(14) |
| | S | | | |
| Frame 3 | GGGTFMDVLHVICEKPHVEHCGS | (11)VIQEIKPH(22) | (3)GTFMDVLHV(21) | (7)DVLHVIQEI(19) |
| No. 20 | | | | |
| Frame 1 | GGGGEGAAQ | GGGGEGAAQ(6) | no data | no data |
| Frame 2 | GEVERECHSEISLLIPANKVLLFSFLSLLSLP | (12)SLLIPANKV(28) | (21)LLFSFLSLL(28) | (13)LLIPANKVLL(28) |
| Frame 3 | GRWRGSSTVKSLC | (1)GRWRGSSTV(16) | (4)RGSSTVKSL(15) | (3)WRGSSTVKSL(15) |
| No. 21 | | | | |
| Frame 1 | VGG | no data | no data | no data |
| Frame 2 | WEVENS | no data | no data | no data |
| Frame 3 | GRLRILDLGHSRSTAQVTVAFL | (13)STAQVTVAFL(24) | (14)TAQVTVAFL(20) | (5)ILDLGHSRST(18) |
| No. 22 | | | | |
| Frame 1 | WGQVDLF | no data | no data | no data |
| Frame 2 | GGRWIFFKRLLIAQLIRRCVHFHSITIVFIS | (11)LIAQLIRRCV(22) | (4)WIFFKRLLI(18) | (7)FKRLLIAQL(18) |
| Frame 3 | GAGGSFLSACLSLS | (5)SFLSACLSL(17) | (6)FLSACLSLS(15) | (4)GSFLSACLSL(15) |
| No. 23 | | | | |
| Frame 1 | RADLLNQCCHFDKVLKDFYSPNLGSIRAGS | (18)FYSPYNLGSI(17) | (19)YSPYNLGSI(15) | (4)LLNQCCHFD(14) |
| Frame 2 | VRTY | no data | no data | no data |
| Frame 3 | CGPTKSVLSF | (2)GPTKSVLSF(7) | (1)CGPTKSVLSF(7) | (1)CGPTKSVLS(3) |

| | HLA-A2 peptide 4 | HLA-A2 peptide 5 | HLA-A2 peptide 6 | HLA-A2 peptide 7 |
|---|---|---|---|---|
| No. 1 | | | | |
| Frame 1 | (25)SPNPVLIRL(19) | (52)GLREDVPAC(19) | (8)SVPKRWREL(18) | (91)PLPQPPGPV(18) |
| Frame 2 | (3)GKGQRACRSA(7) | (9)CRSAGGSFT(6) | (12)AGGSFTDAS(6) | (7)RACRSAGGSF(6) |
| Frame 3 | (16)SRTRARHRL(12) | (3)GRVKERAEAL(12) | (10)EALEGASRT(11) | (6)KERAEALEGA(10) |
| No. 2 | | | | |
| Frame 1 | (11)VCAFSCVAVL(18) | (15)SCVAVLISIL(16) | (6)FAFGPVCAF(14) | (2)SGGTFAFGPV(12) |
| Frame 2 | (1)AQGARLRLDL(13) | (4)ARLRLDLSA(12) | (9)DLSARSPAW(11) | (3)GARLRLDLS(9) |
| Frame 3 | (119)CLFPPCVVSA(25) | (29)SLTLKASSL(24) | (81)DIKREVLRSV(22) | (107)SEAGMVLPGL(22) |
| No. 3 | | | | |
| Frame 1 | (27)RGRPKCATV(17) | (11)VCSGMDLGHV(17) | (14)GMDLGHVMGE(14) | (43)AYVWAFLYL(13) |
| Frame 2 | (8)TLEFAQVWT(14) | (11)FAQVWTWDM(11) | (7)VTLEFAQVW(10) | (4)GEGVTLEFA(9) |
| Frame 3 | no data | no data | no data | no data |
| No. 4 | | | | |
| Frame 1 | (16)AIHVGAAAPV(22) | (33)QLKPGGRLIL(22) | (21)AAAPVVPQAL(21) | (25)VVPQALIDQL(19) |
| Frame 2 | no data | no data | no data | no data |
| Frame 3 | no data | no data | no data | no data |
| No. 5 | | | | |
| Frame 1 | | | | |
| Frame 2 | (8)LSEIPSCAEL(15) | (20)GTSQRPAST(13) | (13)SCAELGRGT(12) | (16)ELGRGTSQR(11) |
| Frame 3 | no data | no data | no data | no data |
| No. 6 | | | | |
| Frame 1 | (22)ACQRGNRRL(14) | (24)QRGNRRLCVL(14) | (21)CACQRGNRRL(14) | (14)RVCSRRVCA(10) |
| Frame 2 | (22)HARGETGACV(18) | (43)GSAEPSRWV(16) | (25)GETGACVSWV(16) | (8)GGLGRVACA(15) |
| Frame 3 | no data | no data | no data | no data |
| No. 7 | | | | |
| Frame 1 | (14)GWGGGAKRPL(13) | (7)RLWKAHEGW(12) | (2)REIRWRLWKA(12) | (10)KAHEGWGGGA(12) |
| Frame 2 | (4)FDGGSGRRT(7) | (8)SGRRTKGGA(7) | (3)RFDGGSGRRT(6) | (7)GSGRRTKGGA(6) |
| Frame 3 | (4)SMAALEGAR(14) | (13)RVGRGSKET(11) | (3)DSMAALEGA(9) | (2)RDSMAALEGA(9) |
| No. 8 | | | | |
| Frame 1 | (3)QSDTLSLNK(2) | (2)EQSDTLSLNK(−1) | no data | no data |
| Frame 2 | no data | no data | no data | no data |
| Frame 3 | no data | no data | no data | no data |
| No. 9 | | | | |
| Frame 1 | no data | no data | no data | no data |
| Frame 2 | (25)HLGVLRGLL(21) | (13)PIALSKAAL(20) | (27)GVLRGLLST(20) | (31)GLLSTASST(19) |
| Frame 3 | (10)AVARSLYPRL(19) | (38)ALPPSVFPS(18) | (34)AQHPALPPSV(17) | (11)VARSLYPRL(17) |
| No. 10 | | | | |
| Frame 1 | (4)RFGTEDALV(12) | (2)ARRFGTEDAL(12) | (11)LVFFCVAVQ(12) | (6)GTEDALVFF(11) |
| Frame 2 | no data | no data | no data | no data |
| Frame 3 | (6)DGGRASVLL(11) | (9)RASVLLCGS(10) | (11)SVLLCGSSE(10) | (8)GRASVLLCG(8) |
| No. 11 | | | | |
| Frame 1 | (2)TLAAREVRKN(18) | (29)LPTQKQASV(16) | (34)QASVSEEEV(16) | (42)VENGFGVGI(16) |
| Frame 2 | no data | no data | no data | no data |
| Frame 3 | no data | no data | no data | no data |
| No. 12 | | | | |
| Frame 1 | (13)LAFSFLLSY(15) | (15)FSFLLSYFL(14) | (4)SNYRHEPPCL(14) | (12)CLAFSFLLS(12) |
| Frame 2 | (7)GMSHRAWPF(9) | (5)ITGMSHRAWP(8) | (1)LRPRITGMS(4) | (10)HRAWPFLFF(4) |
| Frame 3 | no data | no data | no data | no data |
| No. 13 | | | | |
| Frame 1 | no data | no data | no data | no data |
| Frame 2 | (42)LLPTCQHGAV(23) | (65)LVPVFLWLL(21) | (41)VLLPTCQHGA(21) | (50)AVLLLQKHNV(19) |
| Frame 3 | (16)AVMASDFAV(18) | (22)FAVPKFRYL(18) | (3)SASPVTGIPL(18) | (49)VLYFFYKNTM(18) |
| No. 14 | | | | |
| Frame 1 | no data | no data | no data | no data |
| Frame 2 | (26)LMIQLLPDDV(22) | (39)ITSILQIGKL(22) | (47)KLRPREAKRL(22) | (19)RLSSSLALM(20) |
| Frame 3 | (18)TAFPVRWLL(17) | (4)KIMTMLHPK(15) | (16)LQTAFPVRWL(15) | (14)SRLQTAFPV(14) |
| No. 15 | | | | |
| Frame 1 | (7)VYVPFVLATV(21) | (17)MMMMMPSLRL(20) | (25)RLHCEHRSQL(20) | (12)VLATVMMMM(17) |
| Frame 2 | (4)GTWCMFHLC(7) | (3)PGTWCMFHL(6) | (6)WCMFHLCWP(4) | (5)TWCMFHLCWP(4) |
| Frame 3 | (3)LVRGVCSICA(12) | (8)CSICAGHGDL(12) | (6)GVCSICAGH(8) | (11)CAGHGDLCS(7) |
| No. 16 | | | | |
| Frame 1 | (4)AVEKGSRVD(7) | (9)SRVDGEDEE(7) | (5)VEKGSRVDGE(7) | (18)GKEDGREEG(5) |
| Frame 2 | (25)KAKRIQRRV(14) | (24)KKAKRIQRRV(13) | (7)RAPEWTERM(12) | (28)RIQRRVSFP(12) |
| Frame 3 | (4)CGEGLQSGR(3) | (5)GEGLQSGRR(1) | (6)EGLQSGRRG(−1) | no data |
| No. 17 | | | | |
| Frame 1 | (3)SPFYSSLSFL(16) | (11)FLQKESFPMA(16) | (1)RQSPFYSSL(15) | (15)ESFPMAVTV(15) |
| Frame 2 | (7)PPSPFFKKSL(10) | (2)KVLFTPPSF(9) | (5)FTPPSPFFKK(9) | (8)PSPFFKKSL(8) |
| Frame 3 | (5)LLLPLLSSK(23) | (4)FLLLPLLSS(21) | (6)LLPLLSSKRV(21) | (26)VMSTAWLLL(20) |
| No. 18 | | | | |
| Frame 1 | (13)HTADACPGKV(19) | (26)SPVCRTVHTV(19) | (23)STVSPVCRTV(18) | (24)TVSPVCRTV(18) |
| Frame 2 | (2)GRERMALVAL(17) | (5)RMALVALFTQ(16) | (6)MALVALFTQL(16) | (14)QLMRALGRC(15) |
| Frame 3 | (4)KEWLWWLCSH(8) | (5)EWLWWLCSH(5) | (3)GKEWLWWLC(1)_ | no data |
| No. 19 | | | | |
| Frame 1 | (2)GEALLWMCY(4) | (1)RGEALLWMCY(−1) | no data | no data |
| Frame 2 | (24)LMKRMWNQM(14) | (21)ALWLMKRMW(13) | (1)GGRHFYGCV(12) | (17)TTCRALWLM(12) |
| Frame 3 | (10)HVIQEIKPHV(17) | (4)TFMDVLHVI(15) | (2)GGTFMDVLHV(14) | (6)MDVLHVIQEI(14) |
| No. 20 | | | | |
| Frame 1 | no data | no data | no data | no data |
| Frame 2 | (20)VLLFSFLSLL(26) | (14)LIPANKVLL(22) | (23)FSFLSLLSL(19) | (19)KVLLFSFLSL(20) |
| Frame 3 | (2)RWRGSSTVKS(6) | (3)WRGSSTVKS(6) | (2)RWRGSSTVK(2) | (5)GSSTVKSLC(1) |
| No. 21 | | | | |
| Frame 1 | no data | no data | no data | no data |
| Frame 2 | no data | no data | no data | no data |
| Frame 3 | (11)SRSTAQVTV(16) | (4)RILDLGHSR(15) | (7)DLGHSRSTA(15) | (9)GHSRSTAQV(15) |
| No. 22 | | | | |
| Frame 1 | no data | no data | no data | no data |
| Frame 2 | (10)LLIAQLIRR(18) | (14)QLIRRCVHFH(17) | (19)CVHFHSITIV(17) | (12)IAQLIRRCV(16) |
| Frame 3 | (2)AGGSFLSACL(14) | (1)GAGGSFLSA(14) | (3)GGSFLSACL(13) | (1)GAGGSFLSAC(12) |
| No. 23 | | | | |
| Frame 1 | (5)LNQCCHFDKV(14) | (23)NLGSIRAGS(13) | (6)NQCCHFDKV(12) | (14)VLKDFYSPY(12) |
| Frame 2 | no data | no data | no data | no data |
| Frame 3 | no data | no data | no data | no data |

### REFERENCES

Akiyama et al., Gene Ther., 7:2113-2121, 2000.
Alcaraz et al., Cancer Res., 55:3998-4002, 1994.
Allhoff et al., World J. Urol., 7:12-16, 1989.
An et al., Proc. Amer. Assn. Canc. Res., 36:82, 1995.
An et al., Molec. Urol., 2: 305-309, 1998.
Antibodies: A Laboratory Manual, Cold Spring Harbor Laboratory, Cold Spring Harbor Press, Cold Spring Harbor, N.Y., 1988.
Ashley et al., J. Exp. Med., 186:1177-1182, 1997.
Ashton-Richardt, Crit. Rev. Immunol., 25:161-82, 2005.
Babian et al., J. Urol., 156:432-437, 1996.
Badalament et al., J. Urol., 156:1375-1380, 1996.
Baichwal and Sugden, In: Gene Transfer, Kucherlapati (Ed.), Plenum Press, New York, pp 117-148, 1986.
Banchereau and Steinman, Nature, 392:245-252, 1998.
Bangharn et al., J. Mol. Biol. 13: 238-252, 1965.
Barinaga, Science, 271: 1233, 1996.
Bedzyk et al., J. Biol. Chem., 265:18615, 1990
Bell et al., “Gynecological and Genitourinary Tumors,” In: Diagnostic Immunopathology, Colvin, Bhan and McCluskey (Eds.), 2nd edition, Ch. 31, Raven Press, New York, pp 579-597, 1995.
Bellus, J. Macromol. Sci. Pure Appl. Chem., A31(1):1355-1376, 1994.
Benvenisty and Neshif, Proc. Nat. Acad. Sci. USA, 83:9551-9555, 1986.
Berzofsky et al., J. Clinical Invest., 11:1515-1525, 2004.
Bittner et al., Methods in Enzymol, 153:516-544, 1987.
Bonifaz et al., J. Exp. Med., 196:1627-1638, 2002.
Bookstein et al., Science, 247:712-715, 1990a.
Bookstein et al., Proc. Nat'l Acad. Sci. USA, 87:7762-7767, 1990b.
Bova et al., Cancer Res., 53:3869-3873, 1993
Brawn et al., The Prostate, 28:295-299, 1996.
Campbell, In: Monoclonal Antibody Technology, Laboratory Techniques in Biochemistry and Molecular Biology, Burden and Von Knippenberg (Eds.), Vol. 13:75-83, Elsevier, Amsterdam, 1984.
Capaldi et al., Biochem. Biophys. Res. Comm, 76:425, 1977.
Carter and Coffey, In: Prostate Cancer: The Second Tokyo Symposium, J. P. Karr and H. Yamanak (Eds.), Elsevier, N.Y., pp 19-27, 1989.
Carter and Coffey, Prostate, 16:3948, 1990.
Carter et al., Proc. Nat'l Acad. Sci. USA, 87:8751-8755, 1990.
Carter et al., Proc. Nat'l Acad. Sci. USA, 93: 749-753, 1996.
Carter et al., J. Urol., 157:2206-2209, 1997.
Cech et al., Cell, 27:487496, 1981.
Chang et al., Hepatology, 14: 124A, 1991.
Chaudhary et al., Proc. Nat'l Acad. Sci., 87:9491, 1990
Chen and Okayama, Mol. Cell. Biol., 7:2745-2752, 1987.
Chen et al., Clin. Chem., 41:273-282, 1995a.
Chen et al., Proc. Am. Urol. Assn, 153:267 A, 1995.
Chen et al., Int. Immunol., 15:427-435, 2003.
Chinault and Carbon, “Overlap Hybridization Screening: Isolation and Characterization of Overlapping DNA Fragments Surrounding the LEU2 Gene on Yeast Chromosome III,” Gene, 5:111-126, 1979.
Chomczynski and Sacchi, Anal. Biochem., 162:156-159, 1987.
Christensson et al., J. Urol., 150:100-105, 1993.
Coffin, In: Virology, Fields et al. (Eds.), Raven Press, New York, pp 1437-1500, 1990.
Colberre-Garapin et al., J. Mol. Biol., 150:1, 1981.
Colvin et al., Diagnostic Immunopathology, 2nd edition, Raven Press, New York, 1995.
Cooner et al., J. Urol., 143:1146-1154, 1990.
Couch et al., Am. Rev. Resp. Dis., 88:394-403, 1963.
Coupar et al., Gene, 68:1-10, 1988.
Culver et al., Science, 256:1550-1552, 1992.
Davey et al., EPO No. 329 822.
Deamer and Lister, “Liposome Preparation: Methods and Mechanisms,” In: Liposomes, M. Ostro (Ed.), 1983.
Diamond et al., J. Urol., 128:729-734, 1982.
Donahue et al., J. Biol. Chem., 269:8604-8609, 1994.
Dong et al., Science, 268:884-886, 1995.
Drugs R D, 7:197-201, 2006.
Dubensky et al., Proc. Nat. Acad. Sci. USA, 81:7529-7533, 1984.
Dumont et al., J. Immunol., 152:992-1003, 1994.
Elledge et al., Cancer Res. 54:3752-3757, 1994
Esslinger et al., Hum. Gene Ther., 13:1091-1100, 2002.
European Patent Application EPO No. 320 308
Fearon et al., Science, 247:47-56, 1990.
Fechheirner et al., Proc. Natl. Acad. Sci. USA, 84:8463-8467, 1987.
Flutter et al., Cell Mol. Immunol., 1:22-30, 2004.
Fong and Engleman, Annu. Rev. Immunol., 18:245-273, 2000.
Fong et al., J. Immunol., 167:7150-7156, 2001.
Forster and Symons, Cell, 49:211-220, 1987.
Fraley et al., Proc. Natl. Acad. Sci. USA, 76:3348-3352, 1979.
Friedmann, Science, 244:1275-1281, 1989.
Freifelder, In: Physical Biochemistry Applications to Biochemistry and Molecular Biology, 2nd ed., Wm. Freeman and Co., New York, N.Y., 1982.
Frohman, In: PCR Protocols: A Guide to Methods and Applications, Academic Press, New York, 1990.
Gabrilovich et al., Cell Immunol., 170:111-119, 1996.
Gefter et al., Somatic Cell Genet., 3:231-236, 1977.
Gerlach et al., Nature (London), 328:802-805, 1987.
Ghosh-Choudhury et al., EMBO J., 6:1733-1739, 1987.
Gingeras et al., PCT Application WO 88/10315.
Ghosh and Bachhawat, In: Liver Diseases, Targeted Diagnosis and Therapy Using Specific Receptors and Ligands, Wu et al. (Eds.), Marcel Dekker, New York, pp 87-104, 1991.
Goding, In: Monoclonal Antibodies: Principles and Practice, 2nd ed., Academic Press, Orlando, Fla., pp 60-61, 65-66, 71-74, 1986.
Gomez-Foix et al., J. Biol. Chem., 267:25129-25134, 1992.
Gopal, Mol. Cell. Biol., 5:1188-1190, 1985.
Graham et al., J. Gen. Virol., 36:59-72, 1977.
Graham and van der Eb, Virology, 52:456-467, 1973.
Graham and Prevec, In: Methods in Molecular Biology: Gene Transfer and Expression Protocols 7, E.
J. Murray (Ed.), Humana Press, Clifton, N.J., pp 205-225, 1991.
Gregoriadis (ed.), In: Drug Carriers in Biology and Medicine, pp 287-341, 1979.
Grunhaus and Horwitz, Sem. Virol., 3:237-252, 1992.
Guermonprez et al., Springer Semin. Immunopathol., 26:257-271, 2005.
Harland and Weintraub, J. Cell Biol., 101:1094-1099, 1985.
Harris et al., J. Urol., 157:1740-1743, 1997.
Heng et al., Proc. Nat. Acad. Sci. USA, 89: 9509-9513, 1992.
Hermonat and Muzycska, Proc. Nat. Acad. Sci. USA, 81:6466-6470, 1984.
Hersdorffer et al., DNA Cell Biol., 9:713-723, 1990.
Herz and Gerard, Proc. Natl. Acad. Sci. USA, 90:2812-2816, 1993.
Hess et al., J. Adv. Enzyme Reg., 7:149, 1968.
Hitzeman et al., J. Biol. Chem., 255:2073, 1980.
Holland et al., Biochemistry, 17:4900, 1978.
Horoszewicz, Kawinski and Murphy, Anticancer Res., 7:927-936, 1987.
Horwich, et al., J. Virol., 64:642-650, 1990.
Huang et al., Prostate, 23: 201-212, 1993.
Innis et al., In: PCR Protocols, Academic Press, Inc., San Diego Calif., 1990.
Inouye et al., Nucl. Acids Res., 13:3101-3109, 1985.
International Patent Application No. PCT/US2005/032392
Isaacs et al., Cancer Res., 51:4716-4720, 1991.
Isaacs et al., Sem. Oncol., 21:1-18, 1994.
Israeli et al., Cancer Res., 54:1807-1811, 1994.
Jacobson et al., JAMA, 274:1445-1449, 1995.
Johnson et al., In: Biotechnology and Pharmacy, Pezzuto et al., (Eds.), Chapman and Hall, New York, 1993.
Jones, Genetics, 85:12, 1977.
Jones and Shenk, Cell, 13:181-188, 1978.
Joyce, Nature, 338:217-244, 1989.
Kaneda et al., Science, 243:375-378, 1989.
Kato et al., J. Biol. Chem., 266:3361-3364, 1991.
Kent, et al., Genome Res. 12:996-1006 (2002).
Kim and Cech, Proc. Natl. Acad. Sci. USA, 84:8788-8792, 1987.
Kingsman et al., Gene, 7:141, 1979.
Klein et al., Nature, 327:70-73, 1987.
Knutson et al., Curr. Drug Targets Immune Endocr. Metabol. Disord., 5:365-371.
Kohler and Milstein, Nature, 256:495-497, 1975.
Kohler and Milstein, Eur. J. Immunol., 6:511-519, 1976.
Kwoh et al., Proc. Nat. Acad. Sci. USA, 86:1173, 1989.
Landis et al., CA Cancer J. Clin., 48: 6-29, 1998.
Le Gal La Salle et al., Science, 259:988-990, 1993.
Levrero et al., Gene, 10 1: 195-202, 1991.
Liang and Pardee, Science, 257:967-971, 1992.
Liang and Pardee, U.S. Pat. No. 5,262,311, 1993.
Liang et al., Cancer Res., 52:6966-6968, 1992.
Lifton, Science, 272:676, 1996.
Lilja et al., Clin. Chem., 37:1618-1625, 1991.
Lithrup et al., Cancer, 74:3146-3150, 1994.
Lowy et al., Cell, 22:817, 1980.
Macoska et al., Cancer Res., 54:3824-3830, 1994.
Mann et al., Cell, 33:153-159, 1983.
Markowitz et al., J. Virol., 62:1120-1124, 1988.
Marley et al., Urology, 48(6A): 16-22, 1996.
Mayordomo et al., J. Exp. Med., 183:1357-1365, 1996.
McCormack et al., Urology, 45:729-744, 1995.
Michel and Westhof, J. Mol. Biol. 216:585-610, 1990.
Miki et al., Science, 266:66-71, 1994.
Miller et al., PCT Application, WO 89/06700.
Mok et al., Gynecol. Oncol., 52:247-252, 1994.
Morahan et al., Science 272:1811, 1996.
Mulligan et al., Proc. Nat'l Acad. Sci. USA, 78:2072, 1981.
Mulligan, Science, 260:926-932, 1993.
Murphy et al., Cancer, 78: 809-818, 1996.
Murphy et al., Prostate, 26:164-168, 1995.
Nair et al., Int. J. Cancer, 70:706-715, 1997.
Nakamura et al., In: Handbook of Experimental Immunology, (4th Ed.), Weir, E., Herzenberg, L. A., Blackwell, C., Herzenberg, L. (Eds.), Vol. 1, Chapter 27, Blackwell Scientific Publ., Oxford, 1987.
Nicolas and Rubinstein, In: Vectors: A Survey of Molecular Cloning Vectors and Their Uses, Rodriguez and Denhardt (Eds.), Butterworth, Stoneham, p 494-513, 1988.
Nicolau and Sene, Biochim. Biophys. Acta, 721:185-190, 1982.
O'Dowd et al., J. Urol., 158:687-698, 1997.
O'Hare et al., Proc. Nat'l Acad. Sci. USA, 78:1527, 1981.
Oesterling et al., J. Urol., 154:1090-1095, 1995.
Ohara et al., Proc. Nat'l Acad. Sci. USA, 86:5673-5677, 1989.
Orozco et al., Urology, 51:186-195, 1998.
Paglia et al., J. Exp. Med., 183:317-322, 1996.
Parker et al., CA Cancer J. Clin., 65:5-27, 1996.
Partin and Oesterling, Urology, 48 (6A):1-3, 1996.
Partin and Ocstcrling, J. Urol., 152:1358-1368, 1994.
Partin and Oesterling (Eds.), Urology, 48(6A) Supplement:1-87, 1996.
Paskind et al., Virology, 67:242-248, 1975.
PCT Application No. PCT/US87/00880
Patcrsson et al., Clin. Chem., 41(10):1480-1488, 1995.
Piironen et al., Clin. Chem. 42:1034-1041, 1996.
Potter et al., Proc. Nat. Acad. Sci. USA, 81:7161-7165, 1984.
Racher et al., Biotechnology Techniques, 9:169-174, 1995.
Ragot et al., Nature, 361:647-650, 1993.
Ralph and Veltri, Advanced Laboratory, 6:51-56, 1997.
Ralph et al., Proc. Natl. Acad. Sci. USA, 90(22):10710-10714, 1993.
Reinhold-Hurek and Shub, Nature, 357:173-176, 1992.
Renan, Radiother. Oncol., 19:197-218, 1990.
Ribas de Pouplana and Fothergill-Gilmore, Biochemistry, 33:7047-7055, 1994.
Rich et al., Hum. Gene Ther., 4:461-476, 1993.
Ridgeway, In: Vectors: A Survey of Molecular Cloning Vectors and Their Uses, Rodriguez R L, Denhardt D T (Eds.), Butterworth, Stoneham, pp 467-492, 1988.
Rippe et al., Mol. Cell. Biol., 10:689-695, 1990.
Rosenfeld et al., Science, 252:431-434, 1991.
Rosenfeld et al., Cell, 68:143-155, 1992.
Roux et al., Proc. Nat'l Acad. Sci. USA, 86:9079-9083, 1989.
Sager et al., FASEB J., 7:964-970, 1993.
Sambrook et al., (ed.), In: Molecular Cloning, Cold Spring Harbor Laboratory Press, Cold Spring Harbor, N.Y., 1989.
Santerre et al., Gene, 30: 147-156, 1984.
Sarver, et al., Science, 247:1222-1225, 1990.
Scanlon et al., Proc Natl Acad Sci USA, 88:10591-10595, 1991.
Schuler et al., Curr. Opin. Immunol., 15:138-147, 2003.
Siders et al., Mol. Ther., 7:498-505, 2003.
Sidransky et al., Science, 252:706-709, 1991.
Sidransky et al., Cancer Res., 52:2984-2986, 1992.
Silver et al., Clin. Cancer Res., 3:81-85, 1997.
Slamon et al., Science, 224:256-262, 1984.
Slamon et al., Science, 235:177-182, 1987.
Slamon et al., Science, 244:707-712, 1989.
Smith, U.S. Pat. No. 4,215,051.
Soh et al., J. Urol., 157:2212-2218, 1997.
Stenman et al., Cancer Res., 51:222-226, 1991.
Stinchcomb et al., Nature, 282:39, 1979.
Stratford-Perricaudet and Perricaudet, In: Human Gene Transfer, O. Cohen-Haguenauer et al., (Eds.), John Libbey Eurotext, France, pp 51-61, 1991.
Stratford-Perricaudet et al., Hum. Gene. Ther., 1:241-256, 1990.
Sun and Cohen, Gene, 137:127-132, 1993.
Szoka and Papahadjopoulos, Proc. Nat'l. Acad. Sci. USA, 75: 4194-4198, 1978.
Szybalska et al., Proc. Nat'l Acad. Sci. USA, 48:2026, 1962.
Takahashi et al., Cancer Res., 54:3574-3579, 1994.
Taparowsky et al., Nature, 300:762-764, 1982.
Temin, In: Gene Transfer, Kucherlapati R. (Ed.), Plenum Press, New York, pp 149-188, 1986.
Tooze, In: Molecular Biology of DNA Tumor Viruses, 2nd ed., Cold Spring Harbor Laboratory, Cold Spring Harbor, N.Y., 1991.
Top et al., J. Infect. Dis., 124:155-160, 1971.
Tschemper et al., Gene, 10:157, 1980.
Tur-Kaspa et al., Mol. Cell. Biol., 6:716-718, 1986.
U.S. patent application Ser. No. 08/692,787
U.S. Pat. No. 4,196,265
U.S. Pat. No. 4,215,051
U.S. Pat. No. 4,683,195
U.S. Pat. No. 4,683,202
U.S. Pat. No. 4,800,159
U.S. Pat. No. 4,883,750
U.S. Pat. No. 5,354,855
U.S. Pat. No. 5,359,046
Varmus et al., Cell, 25:23-36, 1981.
Veltri et al., J. Cell Biochem., 19(suppl):249-258, 1994.
Veltri et al., Urology, 48: 685-691, 1996.
Veltri et al., Sem. Ural. Oncol., 16:106-117, 1998.
Veltri et al., Urology, 53:139-147, 1999.
Visakorpi et al., Am. J. Pathol., 145:1-7, 1994.
Wagner et al., Science, 260:1510-1513, 1993.
Walker et al., Proc. Nat'l Acad. Sci. USA, 89:392-396, 1992.
Wan et al., Hum. Gene Ther., 8:1355-1363, 1997.
Watson et al., Cancer Res., 54:4598-4602, 1994.
Welsh et al., Nucl. Acids Res., 20:4965-4970, 1992.
Wigler et at, Cell, 11:223, 1977.
Wigler et al., Proc. Nat'l Acad. Sci. USA, 77:3567, 1980.
Wingo et al., CA Cancer J. Clin., 47: 239-242, 1997.
WO 90/07641, filed Dec. 21, 1990.
Wong ct al., Int. J. Oncol., 3:13-17, 1993.
Wu and Wu, J. Biol. Chem., 262: 4429-4432, 1987.
Wu and Wu, Biochemistry, 27: 887-892, 1988.
Wu and Wu, Adv. Drug Delivery Rev., 12: 159-167, 1993.
Wu et al., Genomics, 4:560, 1989.
Yang et al., Proc. Natl. Acad. Sci. USA, 87:9568-9572, 1990.
Yokoda et al., Cancer Res. 52, 3402-3408, 1992.
Zitvogel et al., J. Exp. Med., 183:87-97, 1996.
Zlotta et al, J. Urol., 157:1315-1321, 1997.

1. An isolated nucleic acid that is expressed by human prostate cancer cells, selected from the group consisting of:
(i) a nucleic acid comprising a sequence contained in any one of SEQ ID NOs: 1 to 23;
(ii) a variant of (i), wherein said variant has a nucleic acid sequence that is at least 70% identical to the sequence of (i) when aligned without allowing for gaps; and
(iii) a fragment of (i) or (ii) having a size of at least 20 nucleotides in length.

2. The nucleic acid of claim 1, which comprises the nucleic acid sequence contained in any one of SEQ ID NOs: 1 to 23 or a fragment thereof.

3. A primer mixture that comprises primers that result in the specific amplification of one of the nucleic acid sequences of claim 1.

4. A method of detecting prostate cancer comprising determining whether a human prostate cell sample expresses a target nucleic acid molecule, wherein said target nucleic acid molecule comprises the sequence of a gene or RNA comprising a nucleic acid sequence selected from the group consisting of SEQ ID NOs: 1 to 23 or of a fragment of said gene or RNA having a size of at least 20 nucleotides in length, or a polypeptide encoded by said nucleic acids.

5. The method of claim 4, wherein said method comprises detecting the expression of said target nucleic acid molecule using a nucleic acid sequence that specifically hybridizes thereto.

6. The method of claim 5, wherein said method comprises detecting the expression of said target nucleic acid molecule using primers that result in the amplification thereof.

7. The method of claim 5, wherein the expression of said target nucleic acid molecule is detected by assaying for the polypeptide encoded by said nucleic acid.

8. The method of claim 7, wherein said assay involves the use of a monoclonal antibody or fragment that specifically binds to said polypeptide.

9. The method of claim 8, wherein said assay comprises an ELISA or competitive binding assay.

10. An antigen expressed by human prostate cancer cells, wherein said antigen is selected from the group consisting of:
(i) an antigen encoded by a nucleic acid sequence having at least 90% sequence identity in any one of SEQ ID NOs: 1 to 23;
(ii) an antigen derived from a protein comprising a sequence having at least 90% identity in any one of SEQ ID NOs: 1 to 23; and
(iii) an antigenic fragment of (i) or (ii).

11. A prostate antigen comprising (i) the amino acid sequence encoded by a nucleic acid sequence selected from the group consisting of SEQ ID NOs.: 1 to 23 or (ii) an amino acid sequence derived from SEQ ID NOS.: 1 to 23, or (iii) an antigenic fragment of (i) or (ii).

12. A monoclonal antibody or antigen-binding fragment thereof that specifically binds to a target polypeptide molecule selected from:
(i) a polypeptide encoded by a nucleic acid molecule comprising the sequence of a gene or RNA comprising a sequence selected from the group consisting of SEQ ID NOS.: 1 to 23, or by a fragment of said gene or RNA having a size of at least 20 nucleotides in length, or a polypeptide derived from SEQ ID NOS.: 1 to 23.
(ii) antigen according to claim 10, and
(iii) an antigenic fragment of (i) or (ii).

13. A monoclonal antibody or fragment thereof that specifically binds antigen of claim 11.

14. The antigen of claim 10 which is attached directly or indirectly to a detectable label.

15. The antibody of claim 12 which is attached directly or indirectly to a detectable label.

16. A diagnostic kit for detection of prostate cancer which comprises a nucleic acid (e.g., a DNA) according to claim 1 and a detectable label.

17. A diagnostic kit for detection of prostate cancer which comprises primers according to claim 3 and a diagnostically acceptable carrier.

18. A diagnostic kit for detection of prostate cancer which comprises a monoclonal antibody according to claim 12 and a detectable label.

19. A method for treating prostate cancer, which comprises administering to a subject a therapeutically effective amount of a ligand, wherein said ligand specifically binds a target molecule selected from (i) a gene or RNA comprising a sequence selected from the group consisting of SEQ ID NOS.: 1 to 23, a variant thereof or a fragment of said gene or RNA having a size of at least 20 nucleotides in length, and (ii) a protein or polypeptide encoded by a gene or RNA comprising a sequence selected from the group consisting of SEQ ID NOS.: 1 to 23, a variant thereof or a fragment of said gene or RNA having a size of at least 20 nucleotides in length, or a polypeptide derived from SEQ ID NOS.: 1 to 23.

20. The method of claim 19, wherein the ligand is a ribozyme or antisense oligonucleotide that inhibits the expression of a gene having a DNA sequence selected from the group consisting of SEQ ID NOS.: 1 to 23 or a fragment, or variant thereof, or a polypeptide derived from SEQ ID NOS.: 1 to 23.

21. The method of claim 19, wherein the ligand is directly or indirectly attached to an effector moiety.

22. The method of claim 21, wherein said effector moiety is a therapeutic radiolabel, enzyme, cytotoxin, growth factor, or drug.

23. A method for treating prostate cancer comprising administering to a subject a therapeutically effective amount of an antigen according to claim 10, and optionally an adjuvant that elicits a humoral or cytotoxic T-lymphocyte response to said antigen.

24. A method for treating prostate cancer comprising administering to a subject a therapeutically effective amount of an antigen presenting cell.

25. The method of claim 24, wherein said antigen presenting cell is a dendritic cell.

26. The method of claim 25, wherein the dendritic cell (i) is pulsed or loaded with peptide derived is used as a cellular vaccine to stimulate T cell immunity against the peptide, and thereby against the tumor, or (ii) is modified by an expression construct that encodes the peptide as a cellular vaccine to stimulate T cell immunity against the peptide, and thereby against the tumor.

27. The method of claim 24, further comprising a second or multiple tumor-associated HLA-restricted peptides.

28. A method for treating prostate cancer comprising administering to a subject a therapeutically effective amount of a ligand, which specifically binds to a protein encoded by a gene or RNA comprising a sequence selected from the group consisting of SEQ ID NOS.: 1 to 23 or a fragment, or variant thereof, or a polypeptide derived from SEQ ID NOS.: 1 to 23 optionally directly or indirectly attached to a therapeutic effector moiety.

29. The method of claim 28, wherein said effector moiety is a radiolabel, enzyme, cytotoxin, growth factor, or drug.

30. The method of claim 29 wherein the radiolabel is yttrium.

31. The method of claim 29 wherein the radiolabel is indium.

32. The method of claim 28 wherein said ligand is a monoclonal antibody or fragment thereof.

33. The method of claim 28 wherein said ligand is a small molecule.

34. The method of claim 28 wherein said ligand is a peptide.

35. The method of claim 28, wherein said ligand binds an extracellular domain of said protein.

36. A molecule, selected from:
(i) protein encoded by a gene or RNA comprising a sequence selected from the group consisting of SEQ ID NOS.: 1 to 23; and
(ii) a nucleic acid molecule encoding a polypeptide of (i).

37. The molecule of claim 36, wherein said polypeptide has 8 to 100 amino acids in length.

38. A method for selecting, identifying, screening, characterizing or optimizing biologically active compounds, comprising contacting a candidate compound with a target molecule and determining whether the candidate compound binds said target molecule, wherein said target molecule is selected from (i) a nucleic acid molecule comprising the sequence of a gene or RNA comprising a nucleic acid sequence selected from the group consisting of SEQ ID NOS.: 1 to 23, (ii) a fragment of said gene or RNA having a size of at least 20 nucleotides in length, and (iii) a polypeptide encoded by (i) or (ii) or a polypeptide derived from SEQ ID NOs.: 1 to 23.


SEQUENCE LISTING (23 sequences)

SEQ ID NO: 1 - DNA, 340 bp, Homo sapiens
ctaccgggggaagggtcaaagagcgtgccgaagcgctggagggagcttcacggacgcgagctaggcaccggctcgcctaatccggtactaatccggcttgctgcttcccgtccaggcctcgctcgccatgggggaggtggagatctcggccctggcctacgtgaagatgtgcctgcatgctgcccggtacccacacgccgcagtcaacgggctgtttttggcgccagcgccgcggtctggagaatgcctgtgcctcaccgactgtgtgcccctcttccacagccacctggacctgtccgtcatgttggaggtcgccctcaaccaggtgcctccgttgcat

SEQ ID NO: 2 - DNA, 434 bp, Homo sapiens
ggctcagggggcacgtttgcgtttggacctgtctgcgcgttctcctgcgtggcagtcctgatttccatacttctggagaatccatttcgttaacactgaaagccagttctcttttcctggcagtttttttcattttatttttggcattttttacaagataccgttcgggaaaggcttttgaaaggacggaagcgtattcactgtgcgccagtactcctggctgtgctgtggtttctcccgacgtgcacatcgatctcgtatgtgtggcatctgatattaaacgggaggttttaagaagcgtctgccgtgatcatggagcttcggaagcgggaatggttcttccgggtttgctgttttgtctgtttcccccttgtgtggtttccgcctgcgacagttccagaatttgctctcccactcagtgtgctctgcagctg

SEQ ID NO: 3 - DNA, 328 bp, Homo sapiens
ccaggcagctggcgagggggtcaccctagagtttgctcaggtatggacttgggacatgtgatgggagagtgtgggagccgggggagacccaagtgtgcaacagtggagtgtgtgcttggtgtgtctgcatatgtctgggcatttctttatctgtgacagacacattttattccaacaagcattcattgtagaggccactgtgggtgctggggaatgctgtggggagtaaaattaggcacagttcatgcccttgtatggtgaaacggggagatataaatcaaacatttatgtgatattacttttttctgagagaatctcactccgtcac

SEQ ID NO: 4 - DNA, 376 bp, Homo sapiens
gtaagggatggaagaatgggatatgctgaagaagccccttatgatgccattcatgtgggagctgcagcccctgttgtaccccaggcgctaatagatcagttaaagcccggaggaagattgatattgcctgttggtcctgcaggcggaaaccaaatgttggagcagtatgacaagctacaagatggcagcatcaaaatgaagcctctgatgggggtgatatacgtgcctttaacagataaagaaaagcagtggtccaggtggaagtgattttatcttctgctctttcttcttccacacatgcaaggtgaaagggtgtggattttaagacattagactacaagagctgtttttggttgtcacctttatgctcctcaga

SEQ ID NO: 5 - DNA, 385 bp, Homo sapiens
gtaaaggcatgtggaatccaggggcagctttggctggcacgcaggatccagagtcagctcagctgggctggtactcaggatcagctccatccggtgtgtagggtctagtgtaggggtcagcttggctgggccagggctcagagtcagctcttgcctatgcacaggatccaggttcagctgagtcaggctgggagccaaggtcacctgctgctaggttgcaggtggctcagtgccaggctggaggttaagaccccagtctccaggcagtagcatctcttcagaccacagtggctctcctcatgttgatgctggcctctgggatgttccgcgtcctagctccgcacagctgggtatctcactcagtcgccacctcggcctcccaaca

SEQ ID NO: 6 - DNA, 460 bp, Homo sapiens
gccgaggtcttccctaaggacagggagacttgttgagctcccagtgttgcctgcagctctcgcagggagagcacacttccctccctgagacctccacacgcactacacagatggtcacagccctattctccagccctgcccccgtcagctcatcccagcaccaacccctctcctgggaaacctggattctccttacaactggcggtgcctgtgctgaatgcagctctggagttcagcttcagggaaagaaattcccagctctgaaactatagggctctatcagtgccatggaatccccctcttattcacccaacggcttggttctgctgaaccaatttcccactcctcactgaaccccacccaggacacacaggcgcctgtttcccctctggcatgcacacactcttctggagcacacgcgaccctccctaggccgcctgcttctcggctcccctgcaca

SEQ ID NO: 7 - DNA, 424 bp, Homo sapiens
ccgatcggggaagtttgcagggcaggcggcggcaagttatttaagcactggctcgaagtgaggacagagaactgggcaggaggttacccaggccaattcttgggccattccgccccaaggaacatgggagctgtccccaagctttcttgagccccgccgggcggacgcttctcggctaatctttctgcaacttttacaaagctgcctcctccccagtttgatctctgcaaacacatcctgggcgtccgcggcagattggaaactatttttaccgtctggggccgaggaggtgcattgaaataaggaaggcgaggcgccttctctagcgtttcaccccggaccaggctgggtcacccagtggtctctttgctcccccgccccacccttcgtgcgccttccagagccgccatcgaatctctcttgc

SEQ ID NO: 8 - DNA, 393 bp, Homo sapiens
gtagcagggcagcagacttgggagaacgcgggggatacatccggcccactggaagaaaagggggagctgtaggatggttactgctggtcaacagcagtgagtacaagggccttactcaaaacagcaagaaagtttacacactatacaaccagagtcaccaacttaaatgtctagaagctcaaggcaggtaacacaagcaggtaaatggcctcatgggcatcatcgacaactggaatgcttaaaggagtggcagggattctgttccagtcaactcaagctacgcagcaaggcaagctcagttgacagaatttcctgtaattcaaaagaaactaaaaattttctatggcttctattgatttatttatttagagacagggtatcgctctgttcccc

SEQ ID NO: 9 - DNA, 155 bp, Homo sapiens
gggcaaaggatgttgcccctgtcctgaaggctgtcgcccgatcgctctatccaaggctgccctggggcagcgtcacctgggggtcctgcgggggcttctcagcacagcatccagcactgccacctagtgtgttcccgtcacgtctcctccccccg

SEQ ID NO: 10 - DNA, 139 bp, Homo sapiens
gtggcgaggcggttcgggacggaggacgcgctagtgttcttctgtgtggcagttcagaatgatggatcaagctagatcagcattctctaacttgtttggtggagaaccattgtcatatacccggttcagcctgctctcc

SEQ ID NO: 11 - DNA, 336 bp, Homo sapiens
gacaccctagctgcaagggaggttagaaaaaatgagggacaggattatcatggtcagcatattacttgggggccagatatatcacttcccacacaaaagcaggcctctgtcagtgaggaagaagtggagaatggatttggggtgggcatctgacagcatttgccacactgccctccccaatcctaatgcaggccagtcaacgcaccacccccactccccacccacactccaacccccacagtcctagggtctctcagggagaccatcggtggaatccataacattctaagaccctgcagtttgtaggcaaaatcaggcttcctttgggtgcccccg

SEQ ID NO: 12 - DNA, 180 bp, Homo sapiens
gttgagacctcgaattacaggcatgagccaccgtgcctggccttttcttttcttttaagctactttttaatatatagtaatgactgttaatatagtatatactatgctattcatcaatgctgtaactttcttagtttcattttctcactcaattgaagtccaggtacccaggtccacacg

SEQ ID NO: 13 - DNA, 288 bp, Homo sapiens
ggaggaggtcggcatcaccagtaacaggaatccctctccctttgaaggcagtgatggccagcgactttgcagtgccgaaattccgatacctggagaggctcttgattcttcacgggcattggtgctactcccgacttgccaacatggtgctgtacttcttctacaaaaacacaatgttcgtgggcctcctgttttggttccagtttttctgtggcttctctgcatctaccatgattgaccagtggtatctaatcttctttaatctgctcttctcgtcacctccaacga

SEQ ID NO: 14 - DNA, 203 bp, Homo sapiens
cggaccaggacaaaatcatgacgatgctccatcccaagccttccaggttacaaaccgcctttccagttcgctggctcttatgatccagctcctgcccgacgatgtggacactgtcatcacctccattttacagataggaaagctgaggcccagagaagcgaagcgactgtgtctgtccaagaccacgcgccctcctccctgtc

SEQ ID NO: 15 - DNA, 139 bp, Homo sapiens
cggcgcacctggtacgtggtgtatgttccatttgtgctggccacggtgatgatgatgatgatgccatctctccggctacactgcgagcacagatcacagctctctgtagctgatgctggctctacccactgtattcggg

SEQ ID NO: 16 - DNA, 122 bp, Homo sapiens
ggggaggcaccccaggaaaactcacccttctctggatcctctttgccttcttctctcccatcctcttttccctcctcatcctctccgtccactctggagcccttctccacagcctcgcaccc

SEQ ID NO: 17 - DNA, 476 bp, Homo sapiens
gccgcgggattgaaacgttatacagcggacccagtgagacaccagctggggtggccaaaggagtgctagtgtcacccctctcctaactccagaaagcacagcttgcagctctaggagagactccttttggttgaggaaaggaaagggaagagtaaagaggattttgtctggcaactgggataccagcccagccacagtaacataaaacaacaagcagatccctgaagtctccattccaggccctagctcctggataacatttctagacccaccttgggccagaaaagaacctgttaccctgaaggaaaaaacaaagtcctggaagaactcaccacctgctgactaaagagcccgtagtccttgaataaacattagtagcagccaggcagtactcatcacagacctagaacagtgacggccatgggaaaagactctttttgaagaaaggagagggaggagtaaaaaggactttgtct

SEQ ID NO: 18 - DNA, 239 bp, Homo sapiens
ggccttaggggcacgaggatgtgaatcgacctactacttaatataatggctgtgagaaaaggcctctttcctttcctttccacttttgctccaccctatcaggagccagaaaggcctgatggtgacaaggtgtgaaccgtgtggacagtcctgcacacagggctcactgtggacaccttcccagggcacgcatcagctgtgtgaacaaagccaccaaagccattctttccctcccccgc

SEQ ID NO: 19 - DNA, 379 bp, Homo sapiens
ggggacaggcagaggttgcactgcactccagcctgggtgacagatcaagattctgtctcaagtaataataatgatgatgatgatgatgatgatgatgatgataataacacaccttgttaatgttaattcactgtgttaaatcaattaactcattttctcataattacccttgaacgagtaagttattgacattctctccatgggaaaaaaaacccttggagaaaggtaacttgaatatggccaaatgggcaatttggatagaactgggatttgaacctaggacatctgattccacatccttttcatgagccacaatgctctacatgtggttttatttcttggatcacatgtaacacatccataaaagtgcctccccccg

SEQ ID NO: 20 - DNA, 214 bp, Homo sapiens
ctcccggccgccatggccgcgggatgtcccgaattcttgtaaaactgctgaacaaactctccagtttctcttaagaagagtcaccaggataggcagtttctcaacatttgtgcttcatggcaagctaaggagagagaggaatgagaaaagtaagactttattagcaggtattagcagagagatttcactgtgctgctccctctccacctccccc

SEQ ID NO: 21 - DNA, 267 bp, Homo sapiens
ggcggcaggtctaatctggcacacttgctaacttctaataaagaatcaaatggtcttccaactcacataatgactaattctaaagcatgcaacctcaaagtactacattctccctgttctagaaatacaaccctagagggccagtcttccttcagcattcaaatattgagtagaacaactgaactgtaaggttcaactaaaggaatgcaactgtgacttgagctgtggaacggctatggcctaagtcaagaattctcaacctcccac

SEQ ID NO: 22 - DNA, 314 bp, Homo sapiens
gttaggaggtagaggggctgagcttgctcagaccttgcagtaaattctgtccctgttgcaggtccagtttggcagggaagggacacccggtataccctccgttttctttacagaactccaggaatctgtggggtacagaggagtgccagcagagactggaggctaagccacggtcctgtcccatctgagctgtacttgctcaacctctggatgtcatttaacttataaatacaatagtgatgctgtgaaaatggacacatctgcggattaactgagcgataagcaagcgcttaaaaaagatccacctgccccca

SEQ ID NO: 23 - DNA, 120 bp, Homo sapiens
gcccccacctgctgcccagttcctctatgatccagctctaatgcttcccaaattataaggtgaataaaaatccttaaggaccttgtcaaaatgacaacactgatttagtaggtccgcacg